SEQUENCE LISTING (50 sequences)

SEQ ID NO: 1 - DNA, 13 bp, Rattus norvegicus
gttcagtgtgaac

SEQ ID NO: 2 - DNA, 13 bp, Rattus norvegicus
tgtcaagatgtcc

SEQ ID NO: 3 - DNA, 13 bp, Rattus norvegicus
ggtcgtcatgact

SEQ ID NO: 4 - DNA, 13 bp, Rattus norvegicus
ggacagcctgccc

SEQ ID NO: 5 - DNA, 13 bp, Rattus norvegicus
ggacacagtgccc

SEQ ID NO: 6 - DNA, 13 bp, Homo sapiens
ggccaggctggtc

SEQ ID NO: 7 - DNA, 13 bp, Homo sapiens
ggtcaggctggtc

SEQ ID NO: 8 - DNA, 12 bp, Homo sapiens
ggtcatttgtcc

SEQ ID NO: 9 - DNA, 13 bp, Homo sapiens
attcaaaatgacc

SEQ ID NO: 10 - DNA, 1550 bp, Artificial Sequence
  Synthetic construct
cgcctgttcagtgtgaaccctatcgatcagtgtcaagatgtccactgagctcgggtcgtcatgactgagcccgggccgggacagcctgcccgccggatccaaggacacagtgccccaaggtacccgcctgttcagtgtgaaccctatcgatcagtgtcaagatgtccactgagctcgggtcgtcatgactgagcccgggccgggacagcctgcccgccggatccaaggacacagtgccccaaggtacccgcctgttcagtgtgaaccctatcgatcagtgtcaagatgtccactgagctcgggtcgtcatgactgagcccgggccgggacagcctgcccgccggatccaaggacacagtgccccaaggtacccgcctgttcagtgtgaaccctatcgatcagtgtcaagatgtccactgagctcgggtcgtcatgactgagcccgggccgggacagcctgcccgccggatccaaggacacagtgccccaaggtaccgcatccatcacactggcggccgctcgagcatgcatctagagggcccaattcgccctatagtgagtcgtattacaattcactggccgtcgttttacccgcctgttcagtgtgaaccctatcgatcagtgtcaagatgtccactgagctcgggtcgtcatgactgagcccgggccgggacagcctgcccgccggatccaaggacacagtgccccaagatcctctagagtcgattttcttctcgaagtctgatgttccaggtggaatgccaactccagttttggatctcgccagtgattggctagggaggggtttggggcgggccttcctagagcgcggaggcttactagaaagctgtggagccagtctagactgtcactatcaggcactgtagtatttactggtgtgaggattctgcctttctgctagagatcctagcgctaccggactcagatctcgagctcaagcttcgaattctgcagtcgacggtaccgcgggcccgggatccaccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtaacttcaagatccgccacaacatcgaggacggcagcgtcagctcg

SEQ ID NO: 11 - DNA, 118 bp, Artificial Sequence
  Synthetic cassette
cgcctgttcagtgtgaaccctatcgatcagtgtcaagatgtccactgagctcgggtcgtcatgactgagcccgggccgggacagcctgcccgccggatccaaggacacagtgccccaa

SEQ ID NO: 12 - DNA, 10 bp, Arabidopsis thaliana
cctggtccca

SEQ ID NO: 13 - DNA, 10 bp, Anopheles gambiae
cctgcttgtc

SEQ ID NO: 14 - DNA, 10 bp, Homo sapiens
ccccctggat

SEQ ID NO: 15 - DNA, 10 bp, Homo sapiens
gcctacccga

SEQ ID NO: 16 - DNA, 10 bp, Homo sapiens
atcgtggcgg

SEQ ID NO: 17 - DNA, 10 bp, Homo sapiens
gacatcaagt

SEQ ID NO: 18 - DNA, 10 bp, Unknown
  SAGE tag
cgaatgtcct

SEQ ID NO: 19 - DNA, 10 bp, Homo sapiens
gcccctgctg

SEQ ID NO: 20 - DNA, 10 bp, Homo sapiens
gtgcggagga

SEQ ID NO: 21 - DNA, 10 bp, Homo sapiens
tgccctcagg

SEQ ID NO: 22 - DNA, 10 bp, Homo sapiens
agaacttcct

SEQ ID NO: 23 - DNA, 10 bp, Homo sapiens
tgtgggaaat

SEQ ID NO: 24 - DNA, 10 bp, Homo sapiens
gcctgcctga

SEQ ID NO: 25 - DNA, 10 bp, Homo sapiens
ttgaatcccc

SEQ ID NO: 26 - DNA, 10 bp, Homo sapiens
agcaggagcg

SEQ ID NO: 27 - DNA, 10 bp, Homo sapiens
taatttgcat

SEQ ID NO: 28 - DNA, 10 bp, Homo sapiens
aggtcctagc

SEQ ID NO: 29 - DNA, 10 bp, Homo sapiens
aggatgaccc

SEQ ID NO: 30 - DNA, 10 bp, Unknown
  SAGE tag
gtgcccgtgc

SEQ ID NO: 31 - DNA, 10 bp, Homo sapiens
cactcaataa

SEQ ID NO: 32 - DNA, 10 bp, Homo sapiens
gatctcttgg

SEQ ID NO: 33 - DNA, 10 bp, Homo sapiens
cctgccccgc

SEQ ID NO: 34 - DNA, 10 bp, Homo sapiens
tgccctcaaa

SEQ ID NO: 35 - DNA, 10 bp, Unknown
  SAGE tag
ctagatagaa

SEQ ID NO: 36 - DNA, 10 bp, Homo sapiens
aagggcgcgg

SEQ ID NO: 37 - DNA, 10 bp, Homo sapiens
ccggccctac

SEQ ID NO: 38 - DNA, 10 bp, Homo sapiens
aaagcacaag

SEQ ID NO: 39 - DNA, 10 bp, Homo sapiens
ctcttcgaga

SEQ ID NO: 40 - DNA, 10 bp, Unknown
  SAGE tag
tctcagattt

SEQ ID NO: 41 - DNA, 10 bp, Unknown
  SAGE tag
gcggcggcgg

SEQ ID NO: 42 - DNA, 10 bp, Homo sapiens
caactggagt

SEQ ID NO: 43 - DNA, 10 bp, Homo sapiens
tctcctggac

SEQ ID NO: 44 - DNA, 10 bp, Homo sapiens
gtcaccccca

SEQ ID NO: 45 - DNA, 10 bp, Homo sapiens
cagctgtccc

SEQ ID NO: 46 - DNA, 10 bp, Homo sapiens
gcccacacag

SEQ ID NO: 47 - DNA, 10 bp, Homo sapiens
gattgaacct

SEQ ID NO: 48 - DNA, 13 bp, Homo sapiens
ggtctcagtgacc

SEQ ID NO: 49 - DNA, 13 bp, Homo sapiens
ggtccatctgaca

SEQ ID NO: 50 - DNA, 13 bp, Homo sapiens
ggtcaccctgtca

(19) United States
(12) Patent Application Publication
(10) Pub. No.: US 2005/0064420 A1
(43) Pub. Date: Mar. 24, 2005

(54) Methods for identifying, isolating, and controlling the growth of estrogen-responsive cells

(75) Inventors: Kornelia Polyak, Brookline, MA (US); Pankaj Seth, Boston, MA (US)

Correspondence Address: FISH & RICHARDSON PC, 225 FRANKLIN ST, BOSTON, MA, 02110, US

(73) Assignee: Dana-Farber Cancer Institute, Inc., Boston, MA (US)

(21) Appl. No.: 10/494,975

(22) PCT Filed: Nov. 8, 2002
(86) PCT No.: PCT/US02/35901

Related U.S. Application Data
(60) Provisional application No. 60/338,136, filed on Nov. 9, 2001.

(51) Int. Cl. C12Q 1/68; G01N 33/574; C07H 21/04; C07K 14/72

(52) U.S. Cl. 435/6; 435/7.23; 435/69.1; 435/456; 435/320.1; 435/325; 530/350; 536/23.2

(57) ABSTRACT

The invention features reporter constructs and reporter vectors useful for the identification and isolation of estrogen-responsive cells. The invention also embraces methods of inhibiting the proliferation or survival of estrogen-responsive breast cancer cells.

### TECHNICAL FIELD

This invention relates to estrogen-responsive cells, and more particularly to identification, isolation, and growth-control of such cells.

### BACKGROUND

Human epidemiological studies and animal models have suggested a role for hormones, and in particular estrogen, in the development of breast cancer [Alberg et al. (1997) Curr. Opin. Oncol. 9:505-511; Nandi et al. (1995) Proc. Natl. Acad. Sci. U.S.A. 3650-3657; Thompson et al. (1998) Carcinogenesis 19:383-386]. Despite the importance of this issue, relatively little is known about the mechanisms that account for the tumorigenic effect of estrogen.

### SUMMARY

The invention is based on the development of a genetic reporter construct that can be used for the identification, or for the isolation, of estrogen-responsive cells. Using such a construct, the inventors isolated normal estrogen-responsive mammary cells and identified genes that were selectively expressed in such cells subsequent to exposure to estrogen. The inventors found that the product of one of these genes (lipocalin 2) (a) inhibits the proliferation or survival of estrogen receptor-expressing, estrogen-dependent breast cancer cells, and (b) enhances the proliferation or survival of some estrogen receptor non-expressing mammary cells. Thus, the invention features the above-described reporter construct, vectors containing the construct, and cells containing the vectors. The invention also includes methods for identifying and isolating estrogen-responsive cells, for inhibiting the growth proliferation and/or survival of estrogen-responsive breast cancer cells, and for enhancing the proliferation or survival of estrogen receptor non-expressing, estrogen-non responsive cells.

More specifically, the invention features a reporter construct that contains: (a) an estrogen response segment that includes five or more estrogen response elements (ERE); (b) a promoter segment containing at least one promoter nucleic acid sequence; and (c) a nucleotide sequence that encodes a reporter polypeptide. The nucleotide sequence is operably linked to the promoter segment and the estrogen response segment. One or more of the EREs can be an ERE from the rat progesterone receptor promoter, the promoter nucleic acid sequence can be the distal promoter of the rat progesterone receptor gene, and the reporter polypeptide can be green fluorescent protein (GFP) (or a functional fragment of GFP) or luciferase (or a functional fragment of luciferase). The estrogen response segment can contain ten or more EREs, e.g., 20 or more EREs or 25 or more EREs.

The invention also embraces a vector containing the reporter construct of the invention, e.g., a plasmid vector or a viral vector such as an adenoviral vector or a replication defective adenoviral vector. Also included in the invention is a cell (e.g., a eukaryotic or a prokaryotic cell) containing the vector of the invention.

Another aspect of the invention is a method of identifying an estrogen-responsive cell. The method involves: (a) introducing a vector of the invention into a test cell; (b) contacting the test cell with estrogen; and (c) determining whether the test cell expresses the reporter polypeptide. Another method of the invention is a method of isolating an estrogen-responsive cell. This method involves: (a) introducing a vector of the invention into a plurality of cells; (b) contacting the cells with estrogen; and (c) isolating a cell that expresses the reporter polypeptide.

The invention also features a method of inhibiting the proliferation or survival of an estrogen-responsive cancer cell in a mammalian subject. The method involves: (a) identifying a mammalian subject as having an estrogen-responsive cancer cell and (b) administering to the subject a lipocalin 2 polypeptide or a DNA that encodes a lipocalin 2 polypeptide. The estrogen-responsive cancer cell can be a breast cancer cell and the mammalian subject can be a human patient. The administering can also involve: (a) providing a recombinant cell that is a progeny of a cell obtained from the mammal and has been transfected or transformed ex vivo with a DNA encoding a lipocalin 2 polypeptide; and ()) administering the cell to the mammal. Another aspect of the invention is an in vitro method of inhibiting the proliferation or survival of an estrogen-responsive cancer cell. The method involves incubating the estrogen-responsive cancer cell in a culture medium comprising an isolated lipocalin 2 polypeptide. The estrogen-responsive cancer cell can be a breast cancer cell.

Also encompassed by the invention is a method of enhancing the proliferation or survival of an estrogen-responsive cancer cell in a mammalian subject. The method involves: (a) identifying a mammalian subject as having a deficit of estrogen-non-responsive normal cells; and (b) administering to the subject a lipocalin 2 polypeptide or a DNA that encodes a lipocalin 2 polypeptide. The administering can involve: (a) providing a recombinant cell that is a progeny of a cell obtained from the mammal subject and has been transfected or transformed ex vivo with a DNA encoding a lipocalin 2 polypeptide; and (b) admnistering the cell to the mammalian subject. In another aspect, the invention provides an in vitro method of enhancing the proliferation or survival of an estrogen-non-responsive cell. The method involves incubating the estrogen-non-responsive cell (e.g. a normal mammary cell) in a culture medium comprising an isolated lipocalin 2 polypeptide.

As used herein, “operably linked” means incorporated into a genetic construct so that expression control sequences effectively control expression of a coding sequence of interest.

As used herein, a “functional fragment” of lipocalin 2 is a fragment of lipocalin 2 that is shorter than the fill-length, mature lipocalin 2 and has at least 5% of the ability of full-length, mature lipocalin 2 to inhibit the proliferation or survival of T47D breast cancer cells tested as described in Example 5. The fragment preferably has at least 10% 10%, 20%, 30%, 40%, 50%, 60%, 70%, 80%, 90%, 95%, 98%, 99% of this ability, and more preferably has at least 100%. Expression vectors contaiig nucleotide sequence encoding fragments of interest can be made and tested for their ability to inhibit the proliferation or survival of T47D (or similar) breast cancer cells measured as described in Example 5.

The term “isolated lipocalin 2 polypeptide”, as used herein, refers to a lipocalin 2 polypeptide which either has no naturally-occurring counterpart or has been separated or purified from components which naturally accompany it, e.g., in tissues such as pancreas, liver, spleen, ovary, testis, muscle, joint tissue, neural tissue, gastrointestinal tissue or tumor tissue, or body fluids such as blood, serum, or urine. Typically, the lipocalin 2 polypeptide is considered “isolated” when it is at least 70%, by dry weight, free from the proteins and other naturally-occurring organic molecules with which it is naturally associated. Preferably, a preparation of a lipocalin 2 polypeptide is at least 80%, more preferably at least 90%, and most preferably at least 99%, by dry weight, the lipocalin 2 polypeptide. Since a lipocalin 2 polypeptide that is chemically synthesized is, by its nature, separated from the components that naturally accompany it, synthetic lipocalin 2 polypeptide is “isolated.” In addition, lipocalin 2, which may be present in culture medium (used, for example, to culture estrogen-responsive cells) due to its presence in mammalian serum (or any other bodily fluid) that the culture medium contains, is not an isolated lipocalin 2 polypeptide.

An isolated lipocalin 2 polypeptide of the invention can be obtained, for example, by extraction from a natural source (e.g., from tissues); by expression of a recombinant nucleic acid encoding the polypeptide; or by chemical synthesis. A lipocalin 2 polypeptide that is produced in a cellular system different from the source from which it naturally originates is “isolated,” because it will necessarily be free of components which naturally accompany it. The degree of isolation or purity can be measured by any appropriate method, e.g., column chromatography, polyacrylamide gel electrophoresis, or HPLC analysis.

As used herein, “prophylaxis” can mean complete prevention of the symptoms of a disease, a delay in onset of the symptoms of a disease, or a lessening in the severity of subsequently developed disease symptoms. “Prevention” should mean that symptoms of the disease (e.g., cancer) are essentially absent. As used herein, “therapy” can mean a complete abolishment of the symptoms of a disease or a decrease in the severity of the symptoms of the disease. As used herein, a “protective” immune response is an immune response that is prophylactic and/or therapeutic.

Unless otherwise defined, all technical and scientific terms used herein have the same meaning as commonly understood by one of ordinary skill in the art to which this invention pertains. In case of conflict, the present document, including definitions, will control. Preferred methods and materials are described below, although methods and materials similar or equivalent to those described herein can be used in the practice or testing of the present invention. All publications, patent applications, patents and other references mentioned herein are incorporated by reference in their entirety. The materials, methods, and examples disclosed herein are illustrative only and not intended to be limiting.

Other features and advantages of the invention, e.g., inhibiting the growth of cancer cells, will be apparent from the following description, from the drawings and from the claims.

### DESCRIPTION OF DRAWINGS

FIG; 1A is a diagram of the reporter construct used to produce the recombinant Ad-25ERE-GFP adenovirus. The construct contains: (i) an estrogen response segment composed of five identical cassettes (depicted as open triangles labeled 1-5) each containing five different estrogen response elements (shown as small filled boxes labeled ERE1-ERE5) derived from the rat progesterone receptor promoter, (ii) the rat progesterone receptor distal promoter (shown by the filled box labeled “PR. promoter”); (iii) a cDNA sequence encoding green fluorescent protein (shown by the filled box labeled “GFP”). The horizontal lines between the boxes represents nucleotide sequence corresponding to sequence from regions between the EREs in the wild-type the rat progesterone promoter, sequence derived from vectors used in the sequential subcloning steps used to generate the reporter construct, or sequence derived from PCR oligonucleotide primers also used in the subcloning procedure. The nucleotide sequence of the construct is shown in FIG. 5A.

FIG. 1B is a series of fluorescence micrographs showing the fluorescence emitted by cells of the estrogen-responsive breast cancer cell lines T47D, ZR75-1, and BT474 that had been infected in the absence of hormone (“UTREATED”) or in the presence of either estrogen (“ESTROGEN”) or tamoxifen (“TAMOXIFEN”) with (i) a control adenoviral vector (Ad-CMV-GFP) in which the GFP coding sequence is under the control of a constitutive promoter, or (ii) the Ad-25ERE-GFP adenoviral vector.

FIG. 1C is a series of fluorescence flow cytometry histograms showing the fluorescence emitted by some of the cell populations shown in FIG. 1B. Profiles for untreated (filled) and estrogen treated cells (unfilled) infected with the Ad-25ERE-GFP adenovirus are shown.

FIG. 1D is a series of fluorescence flow cytometry histograms showing the fluorescence emitted by estrogen-responsive (“ER+”) T47D breast cancer cells and estrogen-non-responsive (“ER−”) HME50 breast cancer cells that had been (i) infected with a control adenoviral vector (Ad-CMV-GFP) in which the GFP coding sequence is under the control of a constitutive promoter, or (ii) infected with the Ad-25ERE-GFP adenoviral vector in the absence of hormone (“Control”) or in the presence of estrogen. The gates used to estimate the proportions of fluorescent cells and these proportions are indicated.

FIG. 1E is a fluorescence flow cytometry histogram showing the fluorescence emitted by a mixture of equal numbers of estrogen-responsive T47D breast cancer cells and estrogen-non-responsive HME50 breast cancer cells. Both cell populations had been infected with the Ad-25ERE-GFP adenovirus and treated with estrogen prior to mixing. The gates set to collect fluorescent (“GFPpos”) and non-fluorescent (“GFPneg”) cells by FACS and the proportion of all the cells in the gated regions are indicated.

FIG. 1F is photograph of an ethidium bromide-stained agarose electrophoretic gel of RT-PCR reactions performed with RNA extracted from the sorted cell populations (“GFPpos” and “GFPneg”) obtained by FACS of the mixture described for FIG. 1E. RNA extracted from T47D and from HME50 cells was subjected to the same RT-PCR analysis. The RT-PCR analysis used PCR primers designed to test for the presence in the RNA samples of Calla (“C”), progesterone receptor (“P”), and β-actin (“A”). HME50 cells express Calla and not the progesterone receptor and T47D cells express the progesterone receptor and not Calla. The positions on the gel of β-actin, Calla, and progesterone receptor (“PR”) are indicated on the right of side of the gel.

FIG. 2A is a pair of fluorescence flow cytometry histograms showing the fluorescence emitted by normal human mammary epithelial cells infected with the Ad-25ERE-GFP adenovirus in the absence (“Untreated”) and presence (“Estrogen treated”) of estrogen. The proportions of cells gated as being fluorescent are indicated.

FIG. 2B is a photograph of a western blot analysis of lysates of estrogen receptor-expressing MCF-7 breast cancer cells (“MCF-7 cells”), the human normal mammary epithelial cells infected with the Ad-25ERE-GFP adenovirus in the presence of estrogen used for the analysis shown in FIG. 2A (“Un-sorted”), and the cells gated as positive in the bottom histogram of FIG. 2A and sorted by FACS (“Sorted GFP+”). The western blots were stained with antibodies specific for estrogen receptor α (“BERα?) and β-tubulin. Staining for the latter was done in order to control for protein loading to the electrophoretic gels used to obtain the blots.

FIG. 2C is a photograph of an ethidium bromide-stained agarose electrophoretic gel of RT-PCR reactions performed with RNA extracted from unsorted human mammary epithelial. cells infected with the Ad-25ERE-GFP adenovirus in the presence of estrogen (“Un-sorted”) and cells sorted as described for FIG. 2B. Data on sorted cells from two separate experiments are shown (“Sorted1 GFP+” and “Sorted2 GFP+”). The RT-PCR analysis used PCR primers designed to test for the presence in the RNA samples of Calla (“Calla/CD10”) and HIN-1. Mammary myoepithelial cells express Calla and not HIN-1 and mammary luminal epithelial cells express HIN-1 and not Calla.

FIG. 3A is a photograph of northern blots showing the expression of the indicated genes in various estrogen-treated, estrogen-receptor expressing breast cancer cell lines (“ER+ Breast cancer cells”) and estrogen-treated organoids isolated from normal breast tissue excised from five different human subjects (“Normal organoids”).

FIG. 3B is a photograph of northern blots showing the expression of lipocalin 2, S100A2, and β-actin RNA in mammary glands from (i) virgin female mice (“V”); (ii) ovariectomized virgin mice (“Ov”); (iii) virgin mice that had been ovariectomized and then treated for 6 hours with estrogen (“Ov+E6h”); (iv) virgin mice that had been ovariectomized and then treated for 12 hours with estrogen (“Ov+E12h”); and (v) lactating mice (“L”).

FIG. 3C is a series of photomicrographs of histological sections of normal human breast tissue analyzed by (i) mRNA in situ hybridization with digotonin-labeled antisense and sense lipocalin 2 and S100A2 riboprobes; and (ii) immunohistochemical staining with an antibody specific for estrogen receptor α (“ERα IHC”). The immunohistochemical analyses were carried out on tissue sections immediately adjacent to those used for the respective in situ mRNA hybridization analyses.

FIG. 3D is a bar graph showing the numbers of colonies that were obtained after drug (hygromycin) selection (for 2 weeks) of estrogen receptor (“ER”) expressing (“Positive”) MCF-7 and T47D breast cancer cells, estrogen receptor non-expressing (“Negative”) BT549 and MTA-MB-435S breast cancer cells, and estrogen receptor non-expressing (“Negative”) MCF10A normal immortalized mammary epithelial cells that had all been transfected with either a control expression vector (“CEP”) or an expression vector containing cDNA encoding human lipocalin 2 fused at its C-terminus to a double hemagglutinin tag (“LIPO”). The experiments were carried out in T25 tissue culture flasks and the data are expressed as the number of “Colonies/T25 flask”.

FIG. 4 is a bar graph showing the number of colonies that were obtained after culturing estrogen receptor expressing 747D breast cancer cells and estrogen receptor non-expressing MCF10A normal immortalized mammary epithelial cells with conditioned medium from CHO cells recombinantly expressing either GFP (“GEP”) or lipocalin 2 (“LIPO”). The experiments were carried out in the wells of 6-well tissue culture plates. Data, which are expressed as “Colonies/well”, from experiments carried out in medium supplemented with 5% fetal bovine serum (“5% FBS”) or 0.2% fetal bovine serum (“0.2% FBS”) are shown.

FIG. 5A is a depiction of the nucleotide sequence (SEQ ID NO:10) of the reporter construct depicted in FIG. 1A. The five cassettes containing the five different BREs are shown in bold and are underlined. The rat progesterone receptor distal promoter sequence is shown in bold italics. Sequence (derived from the pEGFPN1 vector) containing the GFP coding sequence is in plain font and is underlined with dots. Sequence derived from the pZERO vector or from PCR primers used in the subcloning steps is shown in plain italics.

FIG. 5B is a depiction of the nucleotide sequence (SEQ ID NO:11) of the cassette (containing five different ERE) five copies of which are included in the reporter construct depicted in FIG. 1A and FIG. 5A. The sequences of the five different EREs are underlined and labeled (“ERE1”-“ERE5”). Sequences between the EREs are from the rat progesterone receptor gene promoter region that contains the five ERE.

### DETAILED DESCRIPTION

The inventors generated a reporter construct that they subcloned into an adenoviral vector. The resulting replication defective adenovirus (Ad-25ERE-GFP) generated with this construct was used for the identification and subsequent isolation of estrogen-responsive normal mammary cells. This construct contained a green fluorescent protein (GFP)-encoding cDNA sequence operably linked to (a) the distal promoter of the rat progesterone receptor gene and (b) a concatamer of five cassettes, each cassette containing five estrogen elements from the rat progesterone receptor promoter. Experiments with various breast cancer cell lines infected with Ad-25ERE-GFP indicated that (1) GFP was only expressed in the presence of both estrogen receptor (ER) and either estrogen or tamoxifen and (2) estrogen-responsive cells could be separated from estrogen-non-responsive cells based on the fluorescence emitted by estrogen-responsive cells infected with Ad-25ERE-GFP and exposed to estrogen.

Using this reporter vector, the inventors isolated normal estrogen-responsive mammary cells and by SAGE identified genes selectively expressed in such cells. Northern blot analysis confirmed this selective pattern of expression for some of the genes identified by the SAGE analysis and indicated expression of some of the genes in some breast cancer cell lines. One of the genes identified as being over-expressed in normal mammary cells following estrogen-treatment was the lipocalin 2 gene. The inventors found that transfection of estrogen-responsive breast cancer cell lines with an expression vector containing a lipocalin 2-encoding cDNA sequence resulted in decreased proliferation and/or survival of the cells. On the other hand, the proliferation and/or survival of some estrogen-non-responsive cell lines (tumor and normal) was enhanced by the transfection with the same vector. These findings were confirmed in experiments involving addition of exogenous lipocalin 2 to the estrogen-responsive and non-responsive cells.

Reporter Constructs, Reporter Vectors, and Cells Containing Reporter Vectors

The invention features a reporter construct useful for the identification and/or isolation of estrogen responsive cells. The construct contains: (1) an estrogen response segment composed of a plurality (e.g., one or more, two or more, three or more, four or more, five or more, six or more, seven or more, eight or more, nine or more, ten or more, 11 or more, 12 or more, 13 or more, 14 or more, 15 or more, 16 or more, 17 or more, 18 or more, 19 or more, 20 or more, 21 or more, 22 or more, 23 or more, 24 or more, 25 or more, 30 or more, 35 or more, 40 or more, 50 or more, 60 or more, 70 or more, 80 or more, 90 or more, or 100 or more) estrogen-responsive elements (ERE); (2) a promoter segment containing at least one (e.g., at least one, at least two, at least three, at least four, at least five, at least six, at least seven, at least eight, at least nine, at least ten, at least 15, at least 20, or at least 30) nucleic acid sequences with promoter activity, (3) a DNA sequence (DNA) encoding a reporter polypeptide or all or part of an RNA molecule transcribed from such a DNA sequence.

The EREs of the estrogen response segment can be any EREs. Such ERE, which can be from the promoters of any genes that are estrogen receptor targets, are known in the art. They can be from, for example, the vitillogenin gene promoter [McMahon et al. (1999) Proc. Natl.

Acad. Sci. U.S.A. 96:5382-5387], the EIT-6 gene promoter [copending U.S. provisional application No. 60/337,754], the progesterone receptor gene promoter (see Example 1), cathepsin D, cyclin D1, or pS2/trefoil factor 2. They can also be from the promoter regions of any of the other genes listed in Table 1 whose expression was induced by estrogen. The EREs in a given reporter construct can all be the same, they can all be different, or some can be the same. The EREs can be immediately adjacent to each other or they can be separated by non-ERE nucleotide sequence. They can be separated by one or a few (e.g., two, three, four, five, six, seven, eight, nine, or ten) or a greater number (e.g., 20-100, 20-200, 20-400, 20-600, 20-800, or 1000) nucleotides; they can even be separated by 2, 3 or 4 kb. The nucleotides sequences between EREs can be some or all of nucleotide sequence that occurs between EREs in a wild-type gene or it can be a nucleotide sequence that does not occur between EREs in a wild-type gene, e.g., sequence derived from a cloning vector or any other sequence employed in the cloning steps used to generate a construct of interest. The EREs in the construct can be in the same orientation in which they occur in the wild-type gene or in an opposite orientation.

The nucleotide sequences of EREs from the rat progesterone receptor gene that can be used in the reporter constructs of the invention include: GTTCAGTGTGAAC (SEQ ID NO:1); TGTCAAGATGTCC (SEQ ID NO:2); GGTCGTCATGACT (SEQ ID NO:3); GGACAGCCTGCCC (SEQ ID NO:4); and GGACACAGTGCCC (SEQ ID NO:5). Nucleotide sequences of EREs from the EIT-6 gene that can be used in the reporter constructs of the invention include: GGCCAGGCTGGTC (SEQ ID NO:6); GGTCAGGCTGGTC (SEQ ID NO:7); GGTCATTTGTCC (SEQ ID NO:8) and ATTCAAAATGACC (SEQ ID NO:9). Nucleotide sequences of EREs from the lipocalin 2 gene that can be used in the reporter constructs of the invention include: GGTCTCAGTGACC (SEQ ID NO:48); GGTCCATCTGACA (SEQ ID NO:49). The nucleotide sequences of an ERE from the S100A2 gene that can be used in the reporter constructs of the invention is: GGTCACCCTGTCA (SEQ ID NO:50). Other ERE sequences that can be used in the reporter constructs of the invention are shown in Kraus et al. [(1994) Mol. Endocrinol. 8:952-969).

Nucleic acid sequences useful for the promoter segment can be derived from any promoter region. It is important that the promoters not have high constitutive activity in order to avoid a high “background” of reporter polypeptide production in the absence of estrogen. The nucleic sequences can be whole promoter regions or sequences within the promoter regions having promoter activity. Promoter regions and nucleic acid sequences within them having promoter activity (e.g. TATA boxes, Sp1 elements, or CAAT boxes) are known in the art, as are methods for establishing whether an nucleic acid sequence of interest within a promoter region has promoter activity, and particularly, low or no constitutive promoter activity. Promoters of interest include but are not limited to the cytomegalovirus hCMV immediate early gene, the early or late promoters of SV40 adenovirus, the lac system, the trp system, the TAC system, the TRC system, the major operator and promoter regions of phage A, the control regions of fd coat protein, the promoter for 3-phosphoglycerate kinase, the promoters of acid phosphatase, and the promoters of the yeast α-mating factors, the adenoviral E1b minimal promoter, the thymidine kinase minimal promoter, or the progesterone receptor distal promoter.

Useful reporter polypeptides can be any polypeptide whose presence can be detected.

Thus, the polypeptides can be polypeptides that are directly and specifically detectable, e.g., fluorescent polypeptides such as green fluorescent protein (GFP), enzymes that catalyze a reaction that results in a detectable product, e.g., luciferase, or polypeptides for which a specifically binding antibody is available or can be made. The latter polypeptides include fusion proteins containing whole polypeptides or peptide fragments of polypeptides for which a specifically binding antibody is available or can be made, e.g., hemagglutin fragments or immunoglobulin heavy chain constant region fragments. Examples of useful reporter polypeptide are, without limitation, GFP, variants of GFP such as blue fluorescent protein, yellow fluorescent protein, or cyan fluorescent protein, luciferase, β-lactamase, chloramphenicol acetyltransferase (CAT), adenosine deaminase (ADA), aminoglycoside phosphotransferase (neor, G418^{r}), dihydrofolate reductase (DHFR), hygromycin-B-phosphotransferase (HPH), thymidine kinase (TK), β-galactosidase, xanthine guanine phosphoribosyltransferase (XGPRT). The reporter polypeptides can be the full-length polypeptides, fragments of such polypeptides, or either of these but with conservative substitutions at one more positions. Those reporter polypeptides having conservative substitutions will generally contain not more than 100 (e.g., not more than 100, not more than 90, not more than 80, not more than 70, not more than 60, not more than 50, not more than 40, not more than 30, not more than 20, not more than 10, not more than nine, not more than eight, not more than seven, not more than six, not more than five, not more than four, not more than three, or not more than two) conservative substitutions or only one conservative substitution. Conservative substitutions typically include substitutions within the following groups: glycine and alanine; valine, isoleucine, and leucine; aspartic acid and glutamic acid; asparagine, glutamine, serine and threonine; lysine, histidine and arginine; and phenylalanine and tyrosine. Where reporter polypeptides are not wild-type full length polypeptides, all that is required is that the relevant polypeptide be detectable with at least 10% (e.g., at least 20%, at least 30%, at least 40%, at least 50%, at least 60%, at least 70%, at least 80%, at least 90%, or 100% or more) of the efficiency with which the wild-type, full-length polypeptide is detectable.

The promoter segment is generally located “upstream” (i.e., 5′) of and relatively close to (within about 50 to about 100 nucleotides from) the nucleotide sequence encoding the reporter polypeptide. The estrogen response segment can be upstream or downstream of the reporter polypeptide sequence. Where the nucleotide sequence encoding the reporter polypeptide is a genomic sequence, the estrogen response segment can be in an intron of such a coding sequence. It is understood that the estrogen response segment need not be continuous. Thus, in any given reporter construct, for example, one or more ERE can be upstream, one or more ERE can be downstream, and/or one or more ERE can be in an intron of the reporter polypeptide-encoding sequence. In addition, the EREs can be downstream and/or upstream of the promoter segment. ERE can be adjacent to, within a few (e.g., up to 10), or even up to several (e.g., 2, 3, 4, or 5) kb from the reporter polypeptide coding sequence.

The invention also includes vectors (“reporter vectors”) containing the reporter constructs of the invention. Suitable expression vectors for making the reporter vectors of the invention include plasmids and viral vectors such as herpes viruses, retroviruses, vaccinia viruses, attenuated vaccinia viruses, canary pox viruses, adenoviruses and adeno-associated viruses, among others. Preferred vectors are adenoviral vectors. Also embraced by the invention are cells containing the reporter vectors of then invention. Such cells can be mammalian cells, e.g., cancer cells (such as breast cancer cells or cells of any other cancer recited herein) or normal cells such as any of the normal cells recited herein (e.g., normal mammary cells), insect cells, bacterial cells, or fungal, including yeast, cells.

Methods of Identifying and Isolating Estrogen-Responsive Cells

The invention features methods for (i) identifying and (ii) isolating estrogen responsive cells. In the first method, a reporter vector of the invention is introduced into a cell by contacting the cell with a source of the reporter vector, the cell is exposed to estrogen, and the presence of the reporter polypeptide in the cell is tested. In the second method, a reporter vector of the invention is introduced into a plurality of (i.e., two or more) cells by contacting the cells with a source of the reporter vector, the cells are exposed to estrogen, and a cell (or cells) expressing the reporter polypeptide is isolated.

Cells to be identified as estrogen-responsive and to be isolated can be any type of cell that is naturally estrogen-responsive or has by, for example, recombinant methods been rendered estrogen-responsive. They can thus be (a) normal cells, e.g., hemopoietic cells (e.g., bone marrow cells, macrophages, monocytes, dendritic cells, T cells, or B cells), fibroblasts, epithelial cells, endothelial cells, keratinocytes, or muscle cells or (b) cancer cells, e.g., breast cancer, lung cancer, colon cancer, pancreatic cancer, renal cancer, stomach cancer, liver cancer, bone cancer, hematological cancer (e.g., leukemia or lymphoma), neural tissue cancer, melanoma, ovarian cancer, testicular cancer, prostate cancer, cervical cancer, vaginal cancer, or bladder cancer cells. The cells can be from or in a tissue in which a subpopulation is known or suspected to be estrogen-responsive. Thus, they can be from most tissues and organs including, without limitation, mammary, uterine, neural (such as brain), cardiovascular, or skeletal (e.g., bone, cartilage, ligament, or tendon) tissue. Alternatively, the cells can be recombinant cells made estrogen-responsive by, for example, transfection, transformation, infection, or transgenesis with an a nucleic acid molecule (e.g., an expression vector) encoding an estrogen receptor.

The cells can be contacted with a reporter vector of the invention in vivo in animal or in vitro. In vivo methods of contacting involve administering the reporter constructs to the animal either systemically or directly to a tissue of interest. Routes of administration can be can be any of those listed below. In vitro contacting can involve incubating cells (a) adhered, for example, to the walls of a tissue culture flask or microscope slide or (b) in suspension in tissue culture medium with a reporter vector. Alternatively, the cells can be, for example, in a tissue section on a microscope slide, e.g., a “frozen tissue section”, and a solution containing the reporter vector is placed on the tissue section. One in vitro method of achieving uptake of polynucleotides is transfection. Methods of transfection are known in the art and include, without limitation, calcium phosphate, lipofection, electroporation, viral infection, and biolistic gene transfer. Alternatively, liposomes or polymeric microparticles can be used. The cells can be transiently transfected or stably transfection. Whether the contacting with the reporter vector was in vivo or in vitro, stable transfectants are generally selected in vitro by methods known in the art in which the cells are selected on the basis their expression of a drug-resistance gene, e.g., a neomycin- or a hygromycin-resistance gene.

After introduction of a reporter vector into the cells, the cells are contacted with estrogen. If the cells are in an animal (e.g., a human), particularly a female animal, it is not necessary to administer exogenous estrogen to the animal as the animal produces it endogenously. Nevetherless, if desired, estrogen can be administered to the animal by any of the routes listed herein. If the cells are in vitro they can be exposed by addition of the estrogen to the solution (e.g., culture medium) surrounding them. After incubation of the cells with the estrogen, expression of the reporter polypeptide is detected by methods known in the art. The choice of method depends on the nature of the reporter polypeptide. Where the cells are contacted in vivo, detection of the reporter polypeptide can be in vivo or in vitro after removal of the cells from the animal. Thus, for example, where the reporter polypeptide is a fluorescent polypeptide such as GFP and a large number of cells in an organ or tissue of the animal is estrogen responsive, the presence of the reporter polypeptide in an animal can be detected by exposing the animal (or a tissue or organ of interest) to ultraviolet light and looking for green fluorescence with the naked eye. Similarly where the presence of a reporter protein is detected by means of a radiolabeled antibody that binds to it (see below), estrogen-responsive cells can be detected by scanning methods known in the art. Generally, however, the cells contacted in vivo with a reporter vector of the invention will be removed and tested in vitro for the presence of the reporter polypeptide.

Cells expressing a directly detectable reporter polypeptide can be examined microscopically for the presence of the reporter polypeptide. Thus, for example, where the reporter polypeptide is or contains GFP (or a fluorescent fragment of GFP), fluorescence in the cells (e.g., in suspension, adhered to a tissue culture flask wall or a microscope slide, or in a tissue section on a microscope slide) can be observed by fluorescence microscopy. Where the reporter polypeptide is an enzyme that catalyzes a reaction that results in, for example, a colored, fluorescent, or luminescent product, such products can be detected by any of a variety of methods known in the art. Such methods that can be carried out directly on unfixed cells or tissue sections or on cells or tissue sections fixed after carrying out the enzyme reaction Where the reporter polypeptide is detected by means of an antibody that binds to it, detection can by any of a wide variety of methods known in the art (see below). The presence of fluorescence (generated by any of the above-described types of reporter polypeptide) within a cell can also be tested for fluorescence flow cytometry. The above-described methods have the advantage of allowing analysis of single cells for the presence of a reporter polypeptide. In such methods it is possible, for example, by exposing relevant cells to appropriate reagents (e.g., antibodies labeled in any of a number of ways) to test for estrogen responsiveness and one or more other cellular markers or activities in a single cell.

Detection assays (e.g., enzyme assays or antibody-based assays such as ELISA or immunoblot assays) can also be carried out on cell culture supernatants, cell homogenates, cell lysates, or subcellular fractions. In such assays the reporter polypeptide itself (e.g., GFP), a product of an enzyme reaction catalyzed by an enzyme reporter polypeptide (e.g., luciferase or β-galactosidase), or the presence of an antibody (or a secondary reagent (see below)) bound to a reporter polypeptide can be detected by a variety of techniques known in the art (e.g., electrophoretic assays such as polyacrylamide gel electrophoresis (PAGE), sodium dodecyl sulfate PAGE (SDS-PAGE), chromatographic techniques such as high pressure liquid chromatography (HPLC), thin layer chromatography (TLC), spectrometric techniques such as spectrophotometry, fluorometry, or mass spectroscopy, or radiometry). These techniques are based on the physicochemical properties (e.g., molecular weight, charge, hydrophobicity, color, radioactivity, fluorescence, luminescence, or ability to absorb visible or ultraviolet light) of the reporter polypeptide itself, a product of an enzyme reaction mediated by the reporter polypeptide, or the labeling system used in an antibody-based assay. Appropriate labels for use in antibody-based assays include, without limitation, radionuclides (e.g., ¹²⁵I, ¹³¹I, ³⁵S, ³H, ³²P, or ³³P), enzymes (e.g., alkaline phosphatase or horseradish peroxidase), fluorescent tags (e.g., fluorescein, rhodamine, or phycoerythrin), or luminescent moieties (e.g., Qdot™ nanoparticles supplied by the Quantum Dot Corporation, Palo Alto, Calif.).

ELISA can also be carried out using whole cells that either (a) have been fixed after exposure of the cells to the reporter vector and estrogen and/or (b) express the reporter polypeptide on their surfaces.

In antibody (monoclonal or polyclonal) based assays, the antibody itself or a secondary antibody that binds to it can be detectably labeled. Alternatively, the antibody can be conjugated with biotin, and detectably labeled avidin (a polypeptide that binds to biotin) can be used to detect the presence of the biotinylated antibody. Combinations of these approaches (including “multi-layer sandwich” assays) familiar to those in the art can be used to enhance the sensitivity of the methodologies. Other applicable antibody-based assays include, without limitation, quantitative immunoprecipitation or complement fixation assays.

In all the above-described assays, expression of the nucleotide sequence in a reporter vector is measured as a function of protein expression. While this is a preferred method of detecting expression of this nucleotide sequence, it is understood that detection of mRNA produced by transcription of the nucleotide sequence can also be used to detect its expression. In order to detect and/ or measure mRNA levels, test cells can be lysed and the levels of reporter polypeptide mRNA (“reporter mRNA”) in the lysates or in RNA purified or semi-purified from the lysates determined by any of a variety of methods familiar to those in the art. Such methods include, without limitation, hybridization assays using detectably labeled reporter mRNA-specific DNA or RNA probes and quantitative or semi-quantitative RT-PCR methodologies using appropriate gene-specific oligonucleotide primers. Alternatively, quantitative or semi-quantitative in situ hybridization assays can be carried out using, for example, tissue sections or unlysed cell suspensions, and detectably (e.g., fluorescently or enzyme) labeled DNA or RNA probes. Additional methods for quantitating mRNA include the RNA protection assay (RPA) and SAGE.

After contacting of a population cells of interest with a reporter vector of the invention, exposing the cells to estrogen, and, where necessary, performing any of the above-described procedures to detect the presence of the either the reporter polypeptide or reporter mRNA, the cells can be isolated by any of a variety of techniques known in the art. Naturally, where it is desired to isolate viable estrogen-responsive cells, any pre-isolation procedure (as described above) should be non-fixing. For example, appropriately labeled cells in a population (e.g., on the bottom of culture vessel or on microscope slide) can be individually collected with an apparatus such as a micromanipulator and placed in a receptacle of choice, e.g., a well of a tissue culture plate. Alternatively, if the cell is one of a colony of estrogen-responsive cells, the whole colony can be “picked”. In addition, the cells can be allowed to grow (in the presence of estrogen) after carrying out all the reporter vector introduction, estrogen exposure and reporter gene product detection steps and again single cells or colonies picked. However such manual procedures are laborious and have high potential for resulting in samples contaminated with estrogen-non-responsive cells.

Alternative procedures include those based on the properties of a detectable label. Thus, for example, where the estrogen-responsive cells are rendered fluorescent or luminescent by any of the detection systems described above, they can be isolated by means of a fluorescence activated cell sorter (FACS) using methods known in the art. In addition, where such cells express a reporter polypeptide on their surface, they can be isolated by any of a variety of immunoselection procedures known in the art. Naturally such procedures include FACS. They also include those in which an antibody specific for the cell-surface reporter polypeptide is physically bound to a solid surface. The solid surface with the antibody bound is exposed to the solution containing the cells. Cells expressing the reporter polypeptide adhere via the antibody to the solid surface. The solution containing cells not adhering to the solid surface (mostly estrogen-non-responsive cells) is then separated from the solid surface with the cells bound to it. The cells adhering to the solid surface (substantially pure estrogen-responsive cells), if so desired, can then be eluted from it. The solid surface can be, for example, a plastic tissue culture vessel, e.g., a plastic Petri dish Alternatively, it can be small metallic particles that after binding to estrogen-responsive cells are held firmly to the bottom of vessel (e.g., a plastic test tube) containing them and the beads by placing the vessel on a magnet. Cells not adhering to the metallic beads (i.e., mostly estrogen-non-responsive cells) are removed. If it is desired to obtain a highly purified population of estrogen-responsive cells, the metallic particles with the estrogen-responsive cells bound thereto can, after removal of the solution containing the non-adherent cell, be resuspended in fresh solution and the magnetic separation step can be repeated. These enriching steps can be carried out as often as desired. Purity of the cell population can be monitored by any of a number of procedures known in the art, e.g., microscopy or fluorescence flow cytometry. The above-described immunoselection procedures can be adapted for achieving higher yields of estrogen-responsive cells by employing the above-described secondary reagents (e.g., antibodies that bind to immunoglobulin molecules and/or biotin and avidin) by methods that would be obvious to those in the art. Monoclonal estrogen-responsive cell populations can be obtained by any of a variety of methods known in the art, e.g., micromanipulation or limiting dilution cloning.

Using the above procedures, it is possible to obtain cell populations containing estrogen-responsive cells that are at least 40% (e.g., at least 40%, at least 50%, at least 60%, at least 70%, at least 80%, at least 85%, at least 90%, at least 92%, at least 95%, at least 96%, at least 97%, at least 98%, at least 99%, at least 99.5%, at least 99.8%, or even 100%) estrogen-responsive cells.

These methods of the invention can be applied to identifying and isolating estrogen responsive cells from a wide variety of vertebrate species including birds, fisb, reptiles, amphibia (e.g., frogs), and mammals such as humans, non-human primates (e.g., monkeys), sheep, cattle, pigs, goats, dogs, cats, rabbits, mice, rats, guinea pigs, and hamsters. In addition, they can be applied to recombinant bacterial or fungal (including yeast) cells, e.g., those recombinantly expressing an estrogen receptor.

Methods of Regulating Proliferation or Survival of Estrogen-Responsive and Estrogen-Non-Responsive Cells

The methods of the invention involve contacting an estrogen-responsive cancer cell with lipocalin 2, or a functional fragment thereof, in order to inhibit proliferation and/or survival of the cancer cell. An alternative method involves contacting an estrogen-non-responsive cancer or normal cell with lipocalin 2, or a functional fragment thereof, in order to enhance proliferation or survival of the cell. Such polypeptides or functional fragments can have amino acid sequences identical to wild-type lipocalin 2 or they can contain one or more conservative amino acid substitutions. Those lipocalin 2 polypeptides having conservative substitutions will generally contain not more than 100 (e.g., not more than 100, not more than 90, not more than 80, not more than 70, not more than 60, not more than 50, not more than 40, not more than 30, not more than 20, not more than 10, not more than nine, not more than eight, not more than seven, not more than six, not more than five, not more than four, not more than three, not more than two) conservative substitutions or only one conservative substitution. Examples of conservative amino acid substitutions are provided above. The term “lipocalin 2 polypeptide” refers to any of these polypeptides. Appropriate cancer cells to which the methods of the invention can be applied include, without limitation, breast cancer, lung cancer, colon cancer, pancreatic cancer, renal cancer, stomach cancer, liver cancer, bone cancer, hematological cancer (e.g., leukemia or lymphoma), neural tissue cancer, melanoma, ovarian cancer, testicular cancer, prostate cancer, cervical cancer, vaginal cancer, uterine cancer, Fallopian tube, or bladder cancer cells.

The methods can be performed in vitro, in vivo, or ex vivo. In vitro application of lipocalin 2 polypeptides can be useful, for example, in basic scientific studies of tumor cell biology, e.g., studies on signal transduction or cell cycle analysis. In such in vitro methods, the appropriate cells can be incubated for various times with the lipocalin 2 polypeptide at a variety of concentrations. The concentration of the lipocalin 2 polypeptide is generally at least 0.1 ng/ml (e.g., at least 0.1 ng/ml, at least 1 ng/ml, at least 10 ng/ml, at least 100 ng/ml, at least 1 μg/ml, at least 10 μg/ml, at least 100 μg/ml, at least 1 mg/mL or at least 10 mg/ml) Other incubation conditions known to those in art (e.g., temperature or cancer cell concentration) can also be varied. Inhibition or enhancement of cancer cell proliferation or survival can be tested by methods such as those disclosed herein.

The methods of the invention will preferably be in vivo or ex vivo (see below).

Lipocalin 2 polypeptides are generally useful as estrogen-responsive cancer cell (e.g., breast cancer cell) proliferation- and/or survival-inhibiting therapeutics. Methods to determine whether a particular cancer is estrogen-responsive include those disclosed and claimed herein In addition, lipocalin 2 polypeptides can be useful for enhancing the proliferation and/or survival of estrogen-non-responsive cells in a mammalian subject; such treatment would naturally be directed at non-malignant cells. Thus they could be used, for example, in patients in which it is desired to increase cell numbers and/or enhance cell survival, e.g., patients with depleted hemopoietic systems such as patients undergoing chemotherapy or radiation therapy, patients with immunodeficiencies, or patients with autoimmune diseases. The lipocalin 2 polypeptides can be administered to mammalian subjects (e.g., human estrogen-responsive breast cancer patients) alone or in conjunction with such drugs and/or radiotherapy. As used herein, a compound that is “therapeutic” is a compound that causes a complete abolishment of the symptoms of a disease or a decrease in the severity of the symptoms of the disease. “Prevention” should mean that symptoms of the disease (e.g., cancer) are essentially absent.

These methods of the invention can be applied to a wide range of species, e.g., humans, non-human primates, horses, cattle, pigs, sheep, goats, dogs, cats, rabbits, guinea pigs, hamsters, rats, and mice.

In Vivo Approaches

In one in vivo approach, the lipocalin 2 polypeptide itself is administered to the subject. Generally, the lipocalin 2 polypeptide will be suspended in a pharmaceutically-acceptable carrier (e.g., physiological saline) and administered orally, intravenously, subcutaneously, intradermally, intramuscularly, intrathecally, intraperitoneally, intrarectally, intravaginally, intranasally, intragastrically, intratracheally, or intrapulmonarily. The lipocalin 2 polypeptide can be delivered directly to estrogen-responsive tumor cells, e.g., to a tumor or a tumor bed following surgical excision of the tumor, in order to inhibit proliferation or survival of any remaining tumor cells. The lipocalin 2 polypeptide can also be delivered directly to a site at which it is desired to enhance the proliferation or survival of estrogen-non-responsive non-malignant cells, e.g., to tissues of the hemopoietic or immune system in subjects with depleted hemopoetic systems or autoimmune diseases, respectively. The dosage required depends on the choice of the route of administration; the nature of the formulation; the nature of the patient's illness; the subject's size, weight, surface area, age, and sex; other drugs being administered; and the judgment of the attending physician. Suitable dosages are in the range of 0.001-100.0 mg/kg. Wide variations in the needed dosage are to be expected in view of the variety of polypeptides and fragments available and the differing efficiencies of various routes of administration. For example, oral administration would be expected to require higher dosages than administration by intravenous injection. Variations in these dosage levels can be adjusted using standard empirical routines for optimization as is well understood in the art Administrations can be single or multiple (e.g., 2-, 3-, , 6-, 8-, 10-, 20-, 50-,100-, 150-, or more fold). Encapsulation of the polypeptide in a suitable delivery vehicle (e.g., polymeric microparticles or implantable devices) may increase the efficiency of delivery, particularly for oral delivery.

Alternatively, a polynucleotide containing a nucleic acid sequence encoding a lipocalin 2 polypeptide can be delivered to the appropriate cells in a mammal. Expression of the coding sequence can be directed to any cell in the body of the subject. However, expression will preferably be directed to cells in the vicinity of the cells (e.g., tumor cells) whose proliferation or survival it is desired to inhibit or enhance (“target cells”). In certain embodiments, expression of the coding sequence can be directed to the target cells themselves. This can be achieved by, for example, the use of polymeric, biodegradable microparticle or microcapsule delivery devices known in the art.

Another way to achieve uptake of the nucleic acid is using liposomes, prepared by standard methods. The vectors can be incorporated alone into these delivery vehicles or co-incorporated with tissue-specific or tumor-specific antibodies. Alternatively, one can prepare a molecular conjugate composed of a plasmid or other vector attached to poly-L-lysine by electrostatic or covalent forces. Poly-L-lysine binds to a ligand that can bind to a receptor on target cells [Cristiano et al. (1995) J. Mol. Med. 73:479]. Alternatively, tissue specific targeting can be achieved by the use of tissue-specific transcriptional regulatory elements (IRE) which are known in the art Delivery of “naked DNA” (i.e., without a delivery vehicle) to an intramuscular, intradermal, or subcutaneous site is another means to achieve in vivo expression.

In the relevant polynucleotides (e.g., expression vectors), the nucleic acid sequence encoding the lipocalin 2 polypeptide (including an initiator methionine and optionally a targeting sequence) is operatively linked to a promoter or enhancer-promoter combination. Short amino acid sequences can act as signals to direct proteins to specific intracellular compartments. Such signal sequences are described in detail in U.S. Pat. No. 5,827,516, incorporated herein by reference in its entirety.

Enhancers provide expression specificity in terms of time, location, and level. Unlike a promoter, an enhancer can function when located at variable distances from the transcription initiation site, provided a promoter is present. An enhancer can also be located downstream of the transcription initiation site. To bring a coding sequence under the control of a promoter, it is necessary to position the translation initiation site of the translational reading frame of the peptide or polypeptide between one and about fifty nucleotides downstream (3′) of the promoter. The coding sequence of the expression vector is operatively linked to a transcription terminating region. The DF3 enhancer can be particularly useful for expression of a lipocalin 2 polypeptide in normal epithelial cells or malignant epithelial cells (carcinoma cells), e.g., breast cancer cells [see U.S. Pat. Nos. 5,565,334 and 5,874,415].

Suitable expression vectors include any of those listed above.

Polynucleotides can be administered in a pharmaceutically acceptable carrier. Pharmaceutically acceptable carriers are biologically compatible vehicles that are suitable for administration to a human, e.g., physiological saline or liposomes. A therapeutically effective amount is an amount of the polynucleotide that is capable of producing a medically desirable result (e.g., decreased proliferation or survival of cancer cells) in a treated animal. As is well known in the medical arts, the dosage for any one patient depends upon many factors, including the patient's size, body surface area, age, the particular compound to be administered, sex, time and route of administration, general health, and other drugs being administered concurrently. Dosages will vary, but a preferred dosage for administration of polynucleotide is from approximately 10⁶ to 10¹² copies of the polynucleotide molecule. This dose can be repeatedly administered, as needed. Routes of administration can be any of those listed above.

In view of the findings showing that lipocalin 2 enhances the growth of estrogen-non-responsive cancer cells (see Example 5), the invention also includes methods of inhibiting the proliferation and/or survival of estrogen-non-responsive cancer cells (e.g., some breast cancer cells) by administering an inhibitor of lipocalin 2 or a substance that binds to lipocalin 2 (e.g., an antibody that binds to lipocalin 2) to a subject having, or suspected of having, an estrogen-non-responsive cancer. Antibodies that bind to lipocalin 2 can be polyclonal antibodies or monoclonal antibodies. They can be, for example, IgM or IgG (any subclass). As used herein, the term “antibody” refers not only to whole antibody molecules, but also to antigen-binding fragments, e.g., Fab, F(ab′)₂, Fv, and single chain Fv (ScFv) fragments. An ScFv fragment is a single polypeptide chain that includes both the heavy and light chain variable regions of the antibody from which the ScFv is derived. Such fragments can be produced, for example, as described in U.S. Pat. No. 4,642,334, which is incorporated herein by reference in its entirety. Also useful are chimeric antibodies. Cancers can be any of those listed herein. Methods to establish whether a given cancer is estrogen-non-responsive can be those described herein or others known in the art Routes of administration, doses, and species to which the methods can be applied are all the same as those disclosed above.

Naturally, antibodies specific for lipocalin 2 and inhibitors of lipocalin 2 activity can also be used to inhibit the proliferation and/or survival in vitro of estrogen-non-responsive cancer cells. Such methods involve culturing such cancer cells with an antibody specific for lipocalin 2 or an inhibitor of lipocalin 2 under conditions analogous to those described above for in vitro application of lipocalin 2 polypeptides per se.

Methods to identify compounds that inhibit the activity of lipocalin 2 include those involving addition of a test compounds to either of the assays described in Example 5. A compound that inhibits the proliferation and/or survival of estrogen-non-responsive breast cancer cells (a) that naturally express a lipocalin 2 polypeptide, (b) that express recombinantly a lipocalin 2 polypeptide or (c) in the presence of an exogenous source of a lipocalin 2 polypeptide is potentially a compound that inhibits the activity of lipocalin 2. Such a compound will preferably not inhibit the proliferation or survival of the estrogen-non-responsive breast cancer cells in the absence of lipocalin 2 or will inhibit it at least two-fold (e.g., at least two-fold, at least three-fold, at least four-fold, at least five-fold, at least six-fold, at least seven-fold, at least eight-fold, at least nine-fold, at least ten-fold, at least 20-fold, at least 40-fold, at least 80-fold, at least 100-fold, at least 500-fold, at least 1,000-fold, or at least 10,000-fold) less efficiently than in the presence of lipocalin 2. Lipocalin 2 polypeptides can also be used to screen for compounds that can interact with lipocalin 2 and potentially thereby inhibit its ability to enhance the proliferation or survival of estrogen-non-responsive cancer cells. One of skill in the art would know how to use standard molecular modeling or other techniques to identify small molecules that would bind to appropriate sites (e.g., allosteric sites) on lipocalin 2. One such example is provided in Broughton (1997) Curr. Opin. Chem. Biol. 1, 392-398.

Also in view of the finding that lipocalin 2 enhances the proliferation and/or survival of estrogen-non-responsive cancer cells, the invention includes a method of diagnosing or predicting the susceptibility of a subject to the development of an estrogen non-responsive cancer, e.g., an estrogen non-responsive breast cancer. Such a method involves measuring the level of lipocalin 2 in a body fluid (e.g., blood, urine, saliva, cerebrospinal fluid, colostrum, or breast milk) or a lavage (e.g., a nipple lavage, a lung lavage, a rectal lavage, a bladder lavage, or a vaginal lavage). A level of lipocalin 2 in the test sample significantly higher than (a) the level of lipocalin 2 in the relevant fluid or lavage from a control individual or (b) the mean level of lipocalin 2 in the relevant fluid or lavage from a control population would indicate that the test subject has or is susceptible to the development of an estrogen-non-responsive cancer, e.g. estrogen-non-responsive breast cancer. Where the subject is, for example, a female human patient suspected of having or of being susceptible to the development of breast cancer, a control individual can be an age-matched woman who does not have or is not considered to be susceptible to the development of breast cancer; a control population can be a group of age-matched women who do not have or are not considered to be susceptible to the development of breast cancer. Thus it is understood that a control value can be obtained from an appropriate control individual or an appropriate group of control subjects.

Lipocalin 2 levels in fluids can be measured by any of a variety of methods of detecting proteins such as those described above for detecting reporter polypeptides, e.g., ELISA or immunoblotting. These methods can be applied to any of the cancers listed herein, provided they are estrogen-non-responsive cancers, and to the any of the species listed herein.

Ex Vivo Approaches

An ex vivo strategy can involve transfecting or transducing cells obtained from the subject with a polynucleotide encoding a lipocalin 2 polypeptide. The transfected or transduced cells are then returned to the subject. The cells can be any of a wide range of types including, without limitation, hemopoietic cells (e.g., bone marrow cells, macrophages, monocytes, dendritic cells, T cells, or B cells), fibroblasts, epithelial cells, endothelial cells, keratinocytes, or muscle cells. Such cells act as a source of the lipocalin 2 polypeptide for as long as they survive in the subject. Alternatively, tumor cells (e.g., any of those listed herein), preferably obtained from the subject but potentially from an individual other than the subject, can be transfected or transformed by a vector encoding a lipocalin 2 polypeptide. The tumor cells, preferably treated with an agent (e.g., ionizing irradiation) that ablates their proliferative capacity, are then introduced into the patient, where they secrete exogenous lipocalin 2.

The ex vivo methods include the steps of harvesting cells from a subject, culturing the cells, transducing them with an expression vector, and maintaining the cells under conditions suitable for expression of the lipocalin 2 polypeptide or functional fragment. These methods are known in the art of molecular biology. The transduction step is accomplished by any standard means used for ex vivo gene therapy including calcium phosphate, lipofection, electroporation, viral infection, and biolistic gene transfer. Alternatively, liposomes or polymeric microparticles can be used. Cells that have been successfully transduced can then be selected, for example, for expression of the coding sequence or of a drug resistance gene. The cells may then be lethally irradiated (if desired) and injected or implanted into the patient.

The invention is illustrated, not limited, by the following examples.

### EXAMPLES

### Example 1

### Materials and Methods

Generation of the Ad-25ERE-GFP Adenovirus

To generate an estrogen responsive, GFP (Green Fluorescence Protein)-expressing adenovirus, a DNA fragment containing five cassettes (each cassette containing five EREs (estrogen responsive elements) from the rat progesterone receptor promoter [Kraus et al. (1994) Mol Endocrin. 8:952-969]) was fused to the 5′ end of the distal promoter of the rat progesterone receptor gene. The resulting regulatory sequence was then placed up-stream of a cDNA sequence encoding GFP (derived from pEGFPN1 (Clonetech, Palo Alto, Calif.)) to create a reporter construct that in turn was cloned into the pShuttle plasmid to create pShuttle-25ERE-GFP. The recombinant adenovirus (Ad-25ERE-GFP) containing the reporter construct was generated using pShuttle-25RGFP and the Ad-Easy system [He et al. (1998) Proc. Natl. Acad. Sci. U.S A. 95:2509-2514].

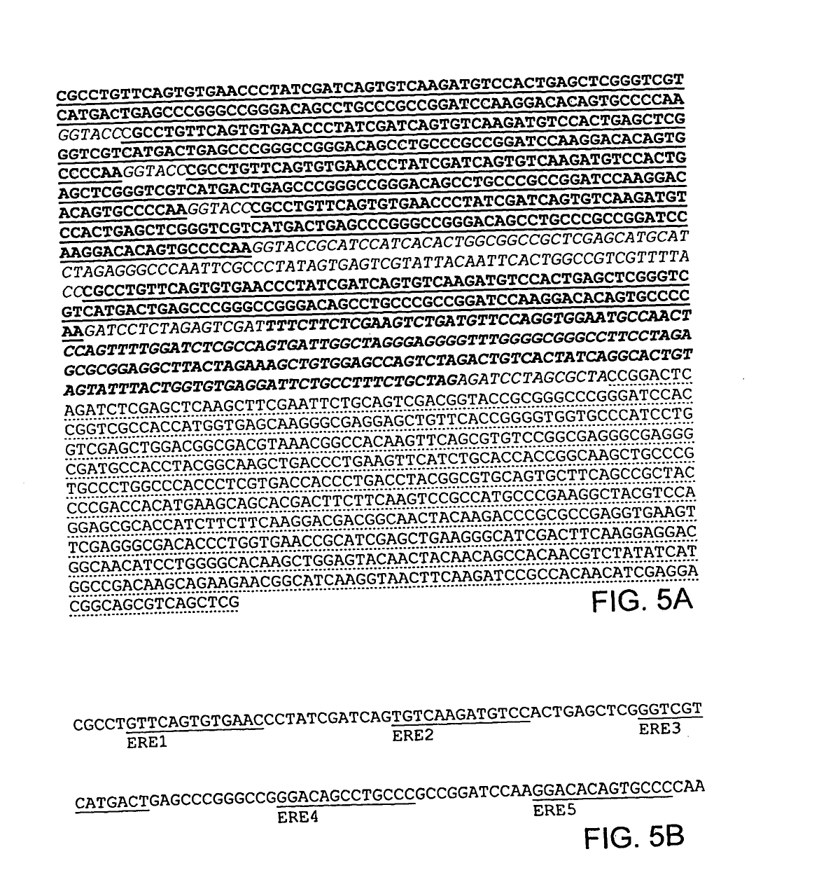

FIG. 5A shows the nucleotide sequence (SEQ ID NO:10) of the reporter construct and FIG. 5B shows the nucleotide sequence (SEQ ID NO:11) of the cassette containing the five EREs from the rat progesterone receptor gene. In the latter, the sequences of the five EREs are underlined.

Cell Culture and Estrogen Treatment

Breast cancer cell lines were obtained from American Type Culture Collection (Manassas, Va.) or were provided by Dr. Marc Lippman (T47D and BT474 cells; University of Michigan, Ann Arbor, Md.) The ME50 myoepithelial cell line was a gift from Dr. J. W. Shay (University of Texas Southwestern Medical Center, Dallas, Tex.) [Shay et al. (1995) Mol. Cell. Biol. 15:425-432]. To assay estrogen responsiveness, cells were cultured in estrogen-free medium (phenol red-free RPMI1640 or DMEM/F12 medium (Life Technologies, Rockville, Md.) supplemented with charcoal/dextran treated fetal bovine serum (cd FBS; 5%) (Hyclone, Logan, Utah)) for 7 days. The cells were tested in fresh estrogen-free medium or fresh estrogen-free medium supplemented with 10 nM estradiol or 10 μM 4-hydroxy-tamoxifen. Cells were collected after 16-24 hours of hormone treatment. Normal mammary cells from reduction mammoplasty tissue were isolated and cultured as described below.

Fluorescence Microscopy and FACS Analysis

For fluorescence microscopy analysis breast cancer cell lines were infected with Ad-25ERE-GFP virus at a MOI (multiplicity of infection) of ˜100 and treated with hormones as described above. Fluorescent micrographs of cells were obtained 48 hours after infection and hormone treatment using a Nikon microscope and a SPOT CCD camera (Diagnostics Instruments, Sterling Heights, MW. For FACS (fluorescence activated cell sorter) analysis, the breast cancer cells were harvested from tissue culture flasks by trypsinization, resuspended in ice cold PBS (phosphate-buffered saline) and analyzed on an Epics flow cytometer (Beckman Coulter, Fullerton, Calif.). For the generation of SAGE libraries ˜100,000 GFP+ cells were sorted by a FACS into DMM/12 medium followed by centrifugation and freezing on dry ice.

Western Blot Analysis

For western blot analysis, cell lysates were resolved by PAGE (polyacrylamide gel electrophoresis), transferred to Immobilon membranes (Millipore, Bedford, Mass.), and stained with monoclonal antibody specific either for human etrogen receptor-α (Ab-11, clone1D5, Neomarkers, Fremont, Calif.) or for human β-tubulin (Ab-3, clone DM1B, Neomarkers).

Generation and Analysis of SAGE Libraries

All human tissue was collected following NIH guidelines and using protocols approved by the Institutional Review Boards (IRB) of the inventors' institutions. Normal human mammary epithelium was derived from 18-24 year-old healthy women undergoing reduction mammoplasty at the Brigham and Women's Hospital, Boston, Mass. Minced breast tissue was digested in phenol-red free DMEM/F12 medium (Life Technologies, Rockville, Md.) supplemented with 1% cdFBS, 2 mg/ml collagenase I (C0130, Sigma), and 2 mg/ml hyaluronidase (H3506, Sigma, St. Luis, Md. at 37° C. for 4-6 hours. Isolated breast ducts (organoids) were collected by centrifugation, filtered through a 100 μm mesh, trypsinized, and resuspended in phenol-red free DMEM/F12 medium (Life Technologies, Rockville, Md.) supplemented with MEGM SingleQuots (Clonetics, Walkersville, Md.) and 20 nM of estradiol. Immediately after plating, the cells were infected with Ad-25ERE-GFP at a MOI (multiplicity of infection) of ˜100. 48 hours later the cells were harvested by trypsinization and analyzed by FACS. GFP expressing (GFP+) cells (˜100,000 cells) were sorted and mRNA was prepared from the GFP+ cells using the SACS kit (Miltenyi Biotec, Germany). The GFP+ cells were also subjected to Western Blot analysis. SAGE libraries were generated and analyzed as previously described [St Croix et al. (2000) Science 289:1197-1202; Porter et al. (2001) Cancer Res. 61:5697-5702]. Hierarchical clustering was applied to data using the Cluster program developed by Eisen et al. [(1998) Proc. Natl. Acad. Sci. U.S.A 95:14863-14868]. Data was filtered for at least 1 observations abs Val 5 and Maxval-Minval>2. By using these settings 4,358 genes (out of 16,808 total) were included in the analysis.

RNA Isolation, RT-PCR, Northern Blot Analysis

RNA isolation, RT-PCR and northern blot analyses were performed as previously described [Polyak et at (1997) Nature 389:300-305; Geng et al. (1999) Cell 97:767-777].

mRNA in Situ Hybridization and Immunohistochemical Analysis

mRNA in situ hybridization using pain sections and digitonin-labeled riboprobes was performed following a protocol developed by St. Croix et al. [(2000) Science 289:1197-1202]. Immunohistochemical analysis of histological sections using antibody specific for the estrogen receptor-α (ERα) (clone 1D5; Dako, Carpinteria, Calif.) was performed as previously described [Polyak et al. (1996) Am. J. Pathol. 149:381-387].

Generation of Lipocalin 2 Mammalian Expression Constructs and Colony Assays

cDNA encoding human lipocalin 2 was amplified by PCR and the isolated PCR product was cloned into the pCEP4 (Invitrogen, Carlsbad, Calif.). The resulting vector (pCEP4-lipocalin-HA) expresses lipocalin-2 with a double hemaglutinnin (HA) tag at its C-terminus. For colony assays, cells were transfected with pCEP4 (control) or pCEP4-lipocalin-HA using FuGene6 (Roche, Indianapolis, Ind.). Transfectants were selected in hygromycin-containing medium for 2 weeks after which colonies were visualized by crystal violet staining. Expression of the lipocalin 2-HA fusion protein was confirmed by western blot analysis (using an antibody specific for HA (Covance, Richmond, Calif.)) of cell lysate and medium from T47D cells transfected with pCEP4 or pCEP4-lipocalin-HA constructs.

For experiments to test for cell growth regulatory activity in secreted lipocalin 2, conditioned medium was generated by infecting COS7 cells with replication-defective adenoviruses expressing either GFP or lipocalin 2. Filtered medium collected 3-4 days after infection was applied to T47 or MCF10A cells. Colonies were visualized by crystal violet staining after seven days of culture.

### Example 2

### Generation and Characterization of the Ad-25ERE-GFP Adenovirus

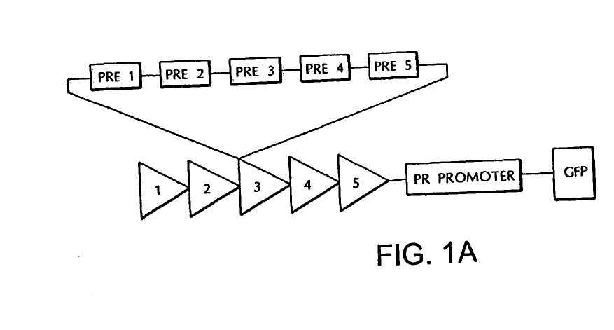

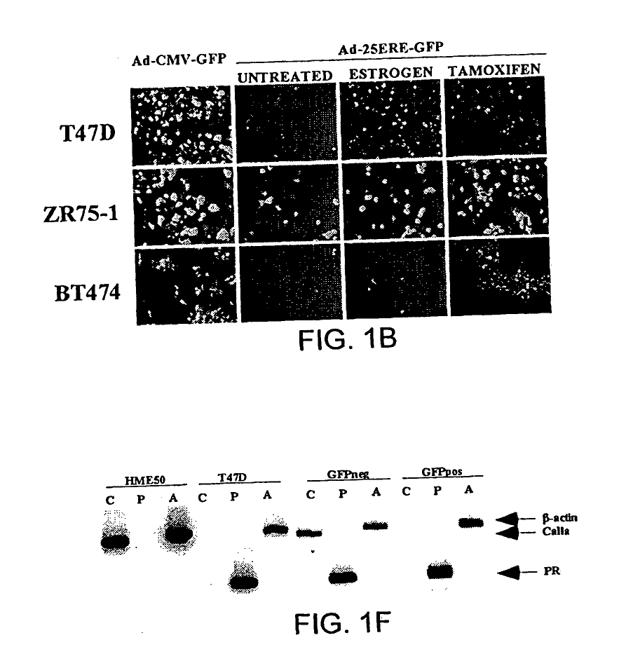

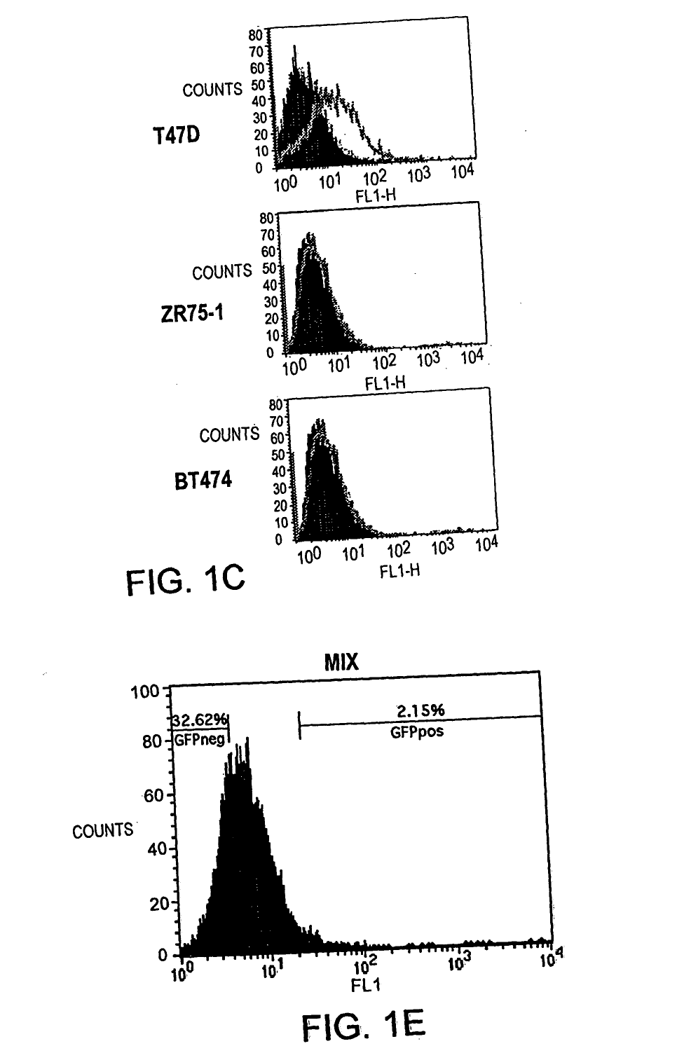

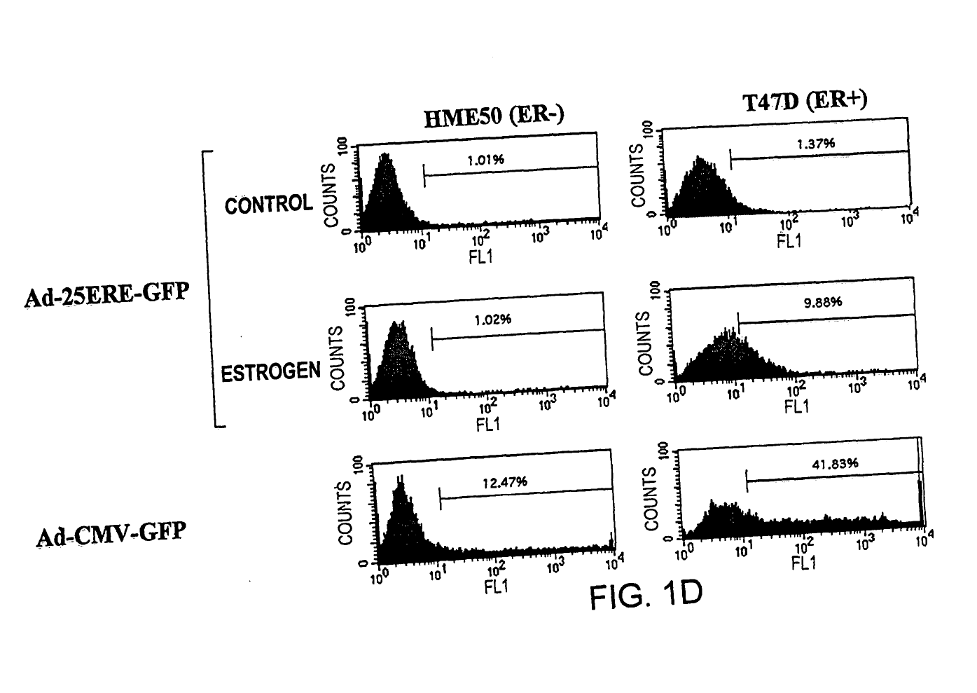

Normal human luminal mammary epithelium contains a small but distinct population of cells that express the ERα. Since ER are not exposed on the surface of cells, a recombinant adenovirus (Ad-25ERE-GFP) that expresses the green fluorescence protein (GFP) gene only in the presence of ER and estrogen was developed for identifying and isolating estrogen-responsive cells (FIG. 1A). Adenoviruses infect mammary epithelial cells with high efficiency, regardless of their proliferation and hormone receptor status [Jeng et al. (1998) Endocrinology 139:2916-2925]. By infecting mammary epithelial cells with the Ad-25ERE-GFP adenovirus, it was possible to isolate ER+ estrogen responsive cells based on GFP expression. Ad-25ERE-GFP was constructed using the promoter region and multimers of estrogen responsive elements (EREs) from the rat progesterone receptor gene, a known in vivo estrogen receptor target [Clarke et al. (1997) Cancer Res. 57:4987-4991]. Ad-25ERE-GFP was then used to infect various estrogen receptor expressing human breast cancer cell lines in the presence of estrogen or tamoxifen or in the absence of hormone (FIG. 1B). In most of the cell lines cultured without hormone there was no significant GFP expression. Addition of estrogen or tamoxifen led to high GFP expression as determined by fluorescence microscopy and FACS (FIGS. 1B and C). Responsiveness to hormone was confirmed by analyzing the level of progesterone receptor, a protein known to be induced in response to estrogen. Infection of estrogen receptor non-expressing (ER−) HME50 myoepithelial cells and BT549 and MDA-MB435S breast cancer cells with Ad-25ERE-GFP did not lead to GFP expression indicating the dependence on the presence of ER for GEP expression (FIG. 1D and data not shown).

Equal numbers of Ad-25ERE-GFP infected and estrogen-treated HME50 (ER−) and Ad-25ERE-GFP infected and estrogen-treated T47D (ER+) cells were mixed. GFP-expressing (GFP+) fractions and GFP-non-expressing (GFP−) fractions of the mixture were obtained by FACS sorting. FIG. 1E shows the relative number of GFP+ and GFP− cells in the mixture; the proportion of cells sorted as GFP+ and GFP− are shown. The two cell types were discriminated by RT-PCR analysis using cell-type-specific genes. HME50 cells express the Calla/CD10 gene [Clarke et al. (1994) Epithelial Cell Biol. 3:3846] but not the progesterone receptor and T47D cells express the progesterone receptor (PR+) but not Calla/CD10 (FIG. 1F). PR+T47D but no Calla/CD10+HME50 cells were in the GFP+ fraction, while both cell types were found in the GFP− population (FIG. 1F). This experiment demonstrated that it was possible to isolate estrogen responsive ER+ cells based on GFP fluorescence from a mixture of estrogen non-responsive ER+ and estrogen un-responsive ER− cells infected with Ad-25ERE-GFP.

### Example 3

### Isolation of Estrogen-Responsive ER+ Cells from Normal Human Mammary Epithelium

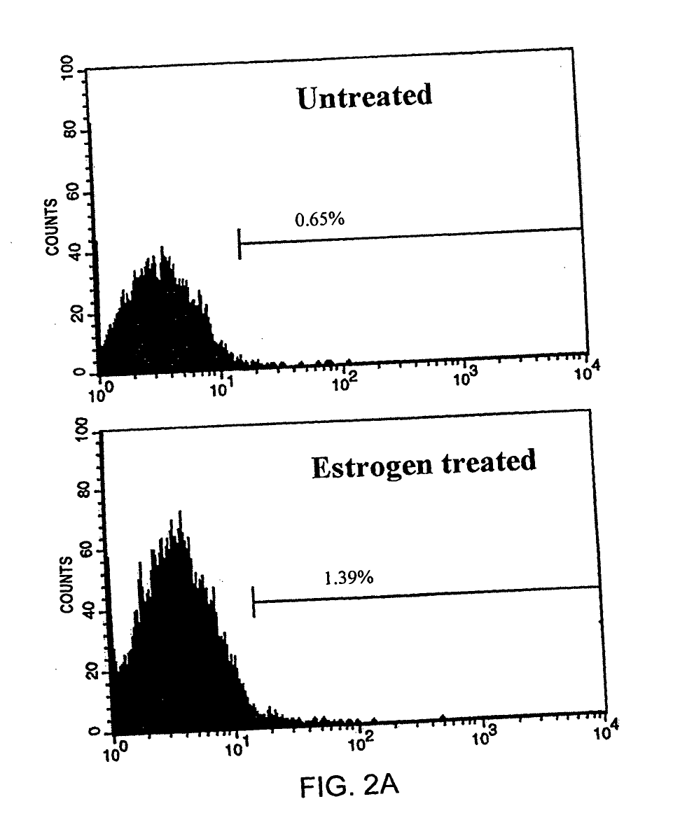

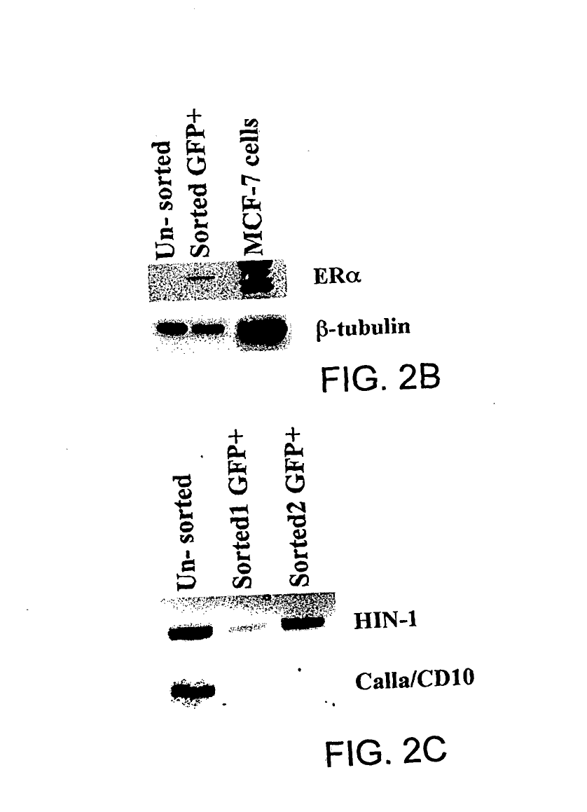

To isolate ER+ estrogen responsive cells from normal human breast tissue, freshly isolated mammary epithelial cells were infected in vitro with Ad-25ERE-GFP in the presence of estrogen. Two days after treatment a small but distinct subpopulation of GEP+ cells was detected in the culture both by fluorescence microscopy (data not shown) and by FACS analysis FIG. 2A). The intensity of GFP fluorescence was much weaker in these cells than in ER+ breast cancer cell lines; this was likely due to the presence of fewer estrogen receptors in normal ER+ cells compared to ER+ breast cancer cells (FIG. 2B). To demonstrate that these GFP+ cells express ERα, an immunoblot analysis of cell extracts from unsorted and GFP+ sorted cells was performed (FIG. 2B). A ˜67 kDa band stained by antibody specific for ERα was detected with GFP+ sorted cells; this band was not detected with unsorted cells. This result confirmed the enrichment of ER+ cells in the GFP+ population. As an additional test of purity, GFP+ cells were analyzed for expression of a myoepithelial marker, ERα-expressing cells are known to have luminal epithelial (and not myoepithelial) features. Unsorted and GFP+ sorted cells from two different experiments were analyzed by RT-PCR using luminal epithelial cell-specific (HIN-1) and myoepithelial cell-specific (Calla/CD10) PCR primers [Clarke et al. (1994) Epithelial Cell Biol. 3:38-46; Krop et al. (2001) Proc. Natl. U.S.A. 98:9796-9801]. GFP+ cells expressed only HIN-1. Thus the GFP+ cells are comprised of luminal epithelial cells while the unsorted fraction was a mixture of luminal epithelial and myoepithelial cells (FIG. 2C).

### Example 4

### Gene Expression Profiles of ER+ Normal and Cancer Cells

In order to determine the identity and abundance of the mRNAs selectively expressed in ER+ estrogen responsive cells from normal mammary epithelium, SAGE libraries from estrogen treated Ad-25ERE-GFP infected and GFP+ cells (normal ER+ cells; NER+ cells) were generated using a modified micro-SAGE protocol [St. Croix et al. (2000) Science 289:1197-1202; Porter et al. (2001) Cancer Res. 61:5697-5702]. Enrichment for luminal epithelial cells in a FACS-sorted GFP+ population of these cells was confirmed by RT-PCR. 34,632 SAGE tags were obtained from the normal ER+ (NER+) cell SAGE library. It was thus possible to analyze the expression levels of close to 14,000 unique individual transcripts.

Since the SAGE tag numbers directly reflect the abundance of the mRNAs, SAGE data obtained from different experiments are directly comparable. Therefore, in order to identify genes only or most abundantly expressed in NER+ cells, the NER+ SAGE library was compared to several other SAGE libraries generated from normal and cancerous breast tissue by the inventors and other investigators [Porter et al. (2001) Cancer Res. 61:5697-5702; Charpentier et al. (2000) Cancer Res. 60:5977-5983]. These libraries included SAGE libraries generated from normal luminal mammary epithelial cells (N1 and N2), ductal carcinoma in situ (D1 and D2), invasive breast carcinomas (I1 and I2), lymph node metastases (M1 and M2), and ER+ breast cancer cell lines (ZR75-1, MCF-7) exposed (ZU and MU) or not exposed to estrogen (ZE and ME3 and MB10) or tamoxifen (ZT). Of the cells used to generate these libraries, the normal luminal epithelial cell (N1 and N2) populations and tumors D1, I1, and M1 contained either only or mostly ER− cells, while tumors D2, I2, M2, and the two cell lines contained only ER+ cells. Based on pair-wise comparisons and statistical analysis 35 transcripts were identified as being only or most abundantly expressed in NER+ cells (Table 1). Unigene database accession numbers are shown in Table 1.

Genes expressed in NER+ cells were of diverse cellular function and almost none of them corresponded to previously characterized estrogen-induced genes. Several of the transcripts were from genes encoding keratins (keratin 5, 6A, 7, 16, and 19), proteases kallikrein 5 and 6), protease inhibitors (SKALP, epididymis-specific whey acidic protein/HE4, and antileukoproteinase), and other secreted (serum amyloid A1, defensin β, heparin-binding growth factor binding protein, lipocalin 2) and membrane associated (MAP17, EMP-1, claudin4, potassium channel, catenin δ1, and GABA-A receptor π subunit) proteins. The expression level of genes encoding several enzymes involved in redox process such as glutathione S-transferase pi, glutathione peroxidase, and sterol-C4-methyl oxidase-like were also significantly elevated in normal ER+ cells. In addition to these known genes, several novel genes with no current database match were identified (Table 1).

TABLE 1
Transcripts specifically or most abundantly expressed
in normal estrogen responsive cells.

| | | | |
|---|---|---|---|
| SEQ | | | |
| ID | UniGene | Gene | SAGE LIBRARIES |

| | | | | | | | | | | | | | | | | |
|---|---|---|---|---|---|---|---|---|---|---|---|---|---|---|---|---|
| SAGE tag | NO: | No: | description | N1 | N2 | D1 | D2 | I1 | M1 | I2 | M2 | ZE | ZU | ME10 | MU | NER |

| | | | | | | | | | | | | | | | | |
|---|---|---|---|---|---|---|---|---|---|---|---|---|---|---|---|---|
| CCTGGTCCA | 12 | 23881 | Keratin 7 | 67 | 52 | 29 | 1 | 21 | 14 | 11 | 0 | 1 | 4 | 4 | 2 | 233 |
| CCTGCTTGC | 13 | 2719 | Epididymis- | 7 | 3 | 0 | 3 | 0 | 0 | 3 | 4 | 0 | 0 | 0 | 0 | 215 |
| | | | specific, whey- | | | | | | | | | | | | | |
| | | | acidic protein | | | | | | | | | | | | | |
| | | | (HE 4) | | | | | | | | | | | | | |
| CCCCCTGGAT | 14 | 183418 | p58/GTA kinase | 33 | 19 | 9 | 5 | 11 | 2 | 8 | 28 | 9 | 48 | 2 | 1 | 183 |
| GCCTACCCGA | 15 | 23582 | Tumor-asso- | 61 | 66 | 11 | 21 | 5 | 1 | 2 | 4 | 9 | 21 | 14 | 6 | 181 |
| | | | ciated | | | | | | | | | | | | | |
| | | | calcium | | | | | | | | | | | | | |
| | | | signal | | | | | | | | | | | | | |
| | | | transducer 2 | | | | | | | | | | | | | |
| ATCGTGGCGG | 16 | 5372 | Claudin-4 | 23 | 74 | 2 | 61 | 1 | 2 | 1 | 2 | 27 | 59 | 1 | 2 | 154 |
| GACATCAAGT | 17 | 182265 | Keratin 19 | 18 | 19 | 32 | 92 | 11 | 5 | 22 | 18 | 21 | 27 | 75 | 67 | 142 |
| CGAATGTCCT | 18 | NA | NO MATCH | 29 | 27 | 0 | 0 | 0 | 0 | 0 | 0 | 0 | 0 | 0 | 0 | 141 |
| GCCCCTGCTG | 19 | 195850 | Keratin 5 | 7 | 11 | 5 | 3 | 0 | 0 | 0 | 0 | 0 | 0 | 0 | 0 | 125 |
| GTGCGGAGGA | 20 | 181062 | Serum | 33 | 11 | 5 | 0 | 2 | 0 | 25 | 0 | 0 | 0 | 0 | 0 | 115 |
| | | | amyloid A1 | | | | | | | | | | | | | |
| TGCCCTCAGG | 21 | 204238 | Lipocalin 2 | 2 | 2 | 0 | 0 | 0 | 0 | 0 | 0 | 2 | 3 | 0 | 0 | 88 |
| | | | (oncogene | | | | | | | | | | | | | |
| | | | 243p3) | | | | | | | | | | | | | |
| AGAACTTCCT | 22 | 32949 | Defensin, | 4 | 0 | 0 | 0 | 0 | 0 | 0 | 0 | 0 | 0 | 0 | 0 | 63 |
| | | | beta 1 | | | | | | | | | | | | | |
| TGTGGGAAAT | 23 | 251754 | Antileuko- | 12 | 13 | 0 | 1 | 0 | 0 | 0 | 0 | 0 | 0 | 0 | 0 | 56 |
| | | | proteinase | | | | | | | | | | | | | |
| | | | (SLPI) | | | | | | | | | | | | | |
| GCCTGCCTGA | 24 | 182506 | Keratin, | 0 | 1 | 1 | 0 | 0 | 0 | 0 | 0 | 0 | 0 | 22 | 12 | 54 |
| | | | hair, basic | | | | | | | | | | | | | |
| | | | 3 | | | | | | | | | | | | | |
| TTGAATCCCC | 25 | 112341 | Protease | 11 | 2 | 0 | 0 | 0 | 0 | 0 | 0 | 0 | 0 | 0 | 0 | 49 |
| | | | inhibitor | | | | | | | | | | | | | |
| | | | 3, skin- | | | | | | | | | | | | | |
| | | | derived | | | | | | | | | | | | | |
| | | | (SKALP) | | | | | | | | | | | | | |
| AGCAGGAGCG | 26 | 70725 | GABA-A | 2 | 2 | 0 | 0 | 0 | 0 | 0 | 0 | 1 | 0 | 0 | 0 | 46 |
| | | | receptor | | | | | | | | | | | | | |
| | | | π subunit | | | | | | | | | | | | | |
| TAATTTGCAT | 27 | 79368 | Epithelial | 4 | 9 | 0 | 1 | 0 | 0 | 5 | 1 | 0 | 0 | 0 | 0 | 44 |
| | | | membrane | | | | | | | | | | | | | |
| | | | protein | | | | | | | | | | | | | |
| | | | 1 (EMP-1) | | | | | | | | | | | | | |
| AGGTCCTAGC | 28 | 226795 | Glutathione | 8 | 11 | 5 | 1 | 23 | 14 | 11 | 0 | 0 | 0 | 0 | 0 | 41 |
| | | | S-transferase | | | | | | | | | | | | | |
| | | | π | | | | | | | | | | | | | |
| AGGATGACCC | 29 | 333418 | HSPC 113 | 1 | 0 | 0 | 0 | 1 | 0 | 0 | 0 | 0 | 2 | 0 | 1 | 34 |
| GTGCCCGTGC | 30 | NA | NO MATCH | 1 | 0 | 0 | 0 | 0 | 0 | 0 | 0 | 0 | 1 | 0 | 0 | 31 |
| CACTCAATAA | 31 | 79361 | Kallikrein | 0 | 3 | 0 | 0 | 0 | 0 | 0 | 0 | 0 | 0 | 0 | 0 | 31 |
| GATCTCTTGG | 32 | 38991 | S100 calcium- | 0 | 1 | 3 | 1 | 0 | 1 | 0 | 0 | 0 | 0 | 0 | 0 | 31 |
| | | | binding | | | | | | | | | | | | | |
| | | | protein A2 | | | | | | | | | | | | | |
| CCTGCCCCGC | 33 | 105039 | Solute | 7 | 1 | 0 | 0 | 0 | 0 | 0 | 0 | 0 | 0 | 0 | 0 | 31 |
| | | | carrier | | | | | | | | | | | | | |
| | | | family 34, | | | | | | | | | | | | | |
| | | | member 2 | | | | | | | | | | | | | |
| TGCCCTCAAA | 34 | 204238 | Lipocalin 2 | 3 | 2 | 0 | 0 | 0 | 0 | 0 | 0 | 0 | 0 | 0 | 0 | 31 |
| | | | (oncogene | | | | | | | | | | | | | |
| | | | 24p3 | | | | | | | | | | | | | |
| CTAGATAGAA | 35 | NA | NO MATCH | 0 | 0 | 0 | 0 | 1 | 0 | 0 | 0 | 0 | 0 | 0 | 0 | 31 |
| AAGGGCGCGG | 36 | 1378 | Annexin A3 | 2 | 5 | 1 | 0 | 0 | 0 | 0 | 0 | 3 | 3 | 0 | 0 | 28 |
| CTCTTCGAGA | 37 | 271473 | MAP 17 | 3 | 3 | 0 | 0 | 0 | 0 | 0 | 0 | 0 | 0 | 0 | 0 | 27 |
| | | | epithelial | | | | | | | | | | | | | |
| | | | protein | | | | | | | | | | | | | |
| AAAGCACAAG | 38 | 334309 | Keratin 6A | 1 | 1 | 0 | 0 | 0 | 0 | 0 | 0 | 0 | 0 | 0 | 0 | 25 |
| CTCTTCGAGA | 39 | 76686 | Glutathione | 7 | 0 | 0 | 1 | 2 | 3 | 1 | 1 | 0 | 0 | 0 | 0 | 24 |
| | | | peroxidase 1 | | | | | | | | | | | | | |
| TCTCAGATTT | 40 | NA | NO MATCH | 0 | 0 | 0 | 0 | 1 | 0 | 0 | 0 | 0 | 0 | 0 | 0 | 12 |
| GCGGCGGCGG | 41 | NA | NO MATCH | 0 | 1 | 0 | 0 | 2 | 1 | 0 | 1 | 5 | 1 | 0 | 0 | 23 |
| CAACTGGAGT | 42 | 166011 | Catenin δ1 | 9 | 0 | 2 | 1 | 0 | 1 | 0 | 0 | 0 | 0 | 0 | 0 | 23 |
| TCTCCTGGAC | 43 | 50915 | Kallikrein 5 | 2 | 1 | 0 | 0 | 0 | 0 | 0 | 0 | 0 | 0 | 0 | 0 | 18 |
| GTCACCCCCA | 44 | 10082 | Potassium | 1 | 0 | 1 | 0 | 0 | 0 | 0 | 0 | 0 | 0 | 0 | 0 | 17 |
| | | | channel, | | | | | | | | | | | | | |
| | | | subfamily N, | | | | | | | | | | | | | |
| | | | member 4 | | | | | | | | | | | | | |
| CAGCTGTCCC | 45 | 115947 | Keratin 16 | 0 | 0 | 0 | 0 | 0 | 0 | 0 | 0 | 0 | 0 | 0 | 0 | 17 |
| GCCCACACAG | 46 | 1690 | Heparin- | 3 | 2 | 0 | 0 | 0 | 0 | 0 | 0 | 0 | 0 | 0 | 0 | 15 |
| | | | binding | | | | | | | | | | | | | |
| | | | growth | | | | | | | | | | | | | |
| | | | factor | | | | | | | | | | | | | |
| | | | binding | | | | | | | | | | | | | |
| | | | protein | | | | | | | | | | | | | |
| GATTGAACCT | 47 | 223018 | Sterol-C4 | 0 | 1 | 1 | 6 | 1 | 0 | 0 | 0 | 0 | 2 | 0 | 0 | 14 |
| | | | methyl | | | | | | | | | | | | | |
| | | | oxidase-like | | | | | | | | | | | | | |

Numbers correspond to SAGE tag numbers observed in the following SAGE libraries: normal luminal epithelial cells (N1 and N2), in situ carcinomas (D1 and D2), invasive carcinomas (I1 and I2), lymph node metastases (M1 and M2), and ER+ breast cancer lines. The breast cancer SAGE libraries were from: ZR75-1 cells treated with estrogen (ZE) and not treated with estrogen (ZU); and from MCF-7 cells treated with estrogen (ME10) and not treated with estrogen (MU).

### Example 5

### Analysis and Function of Normal ER+ Cell Specific Genes

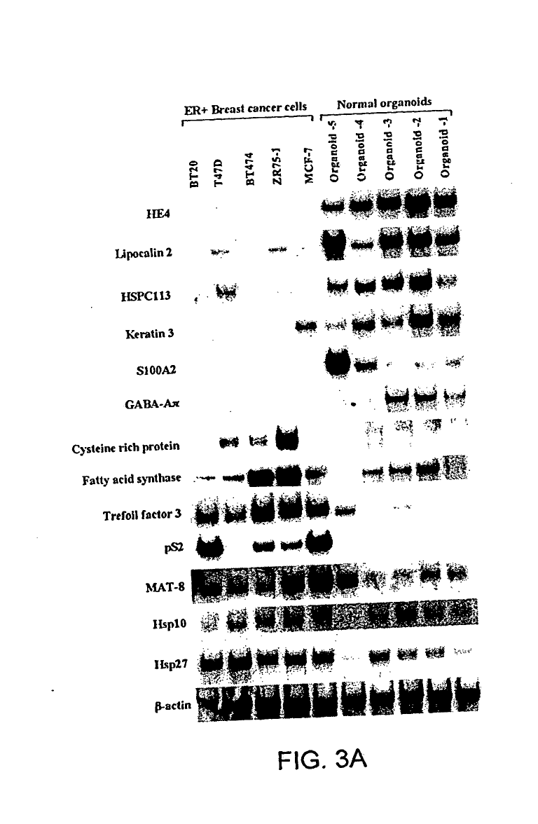

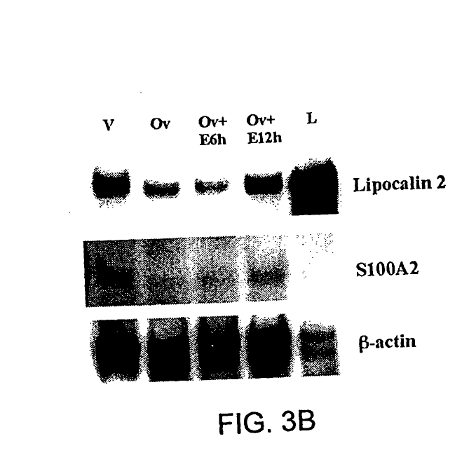

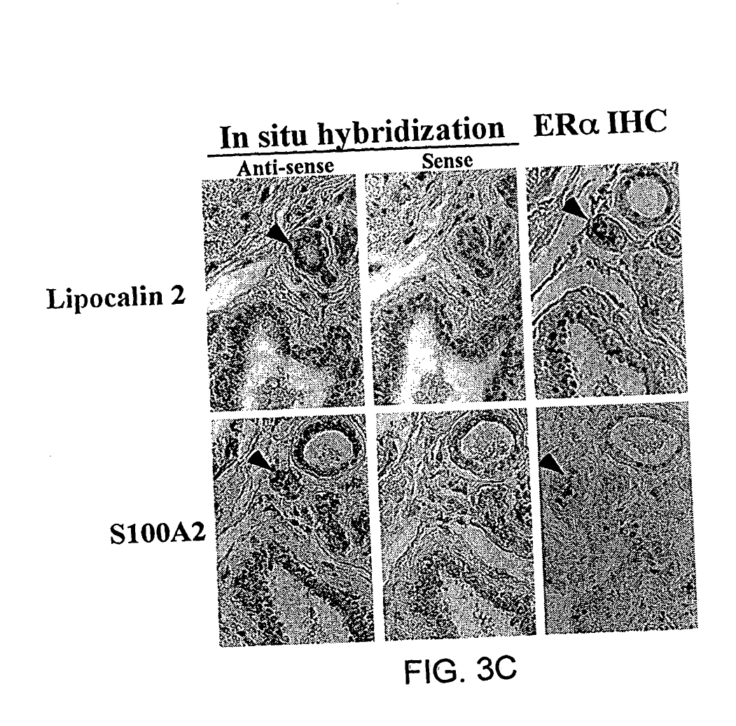

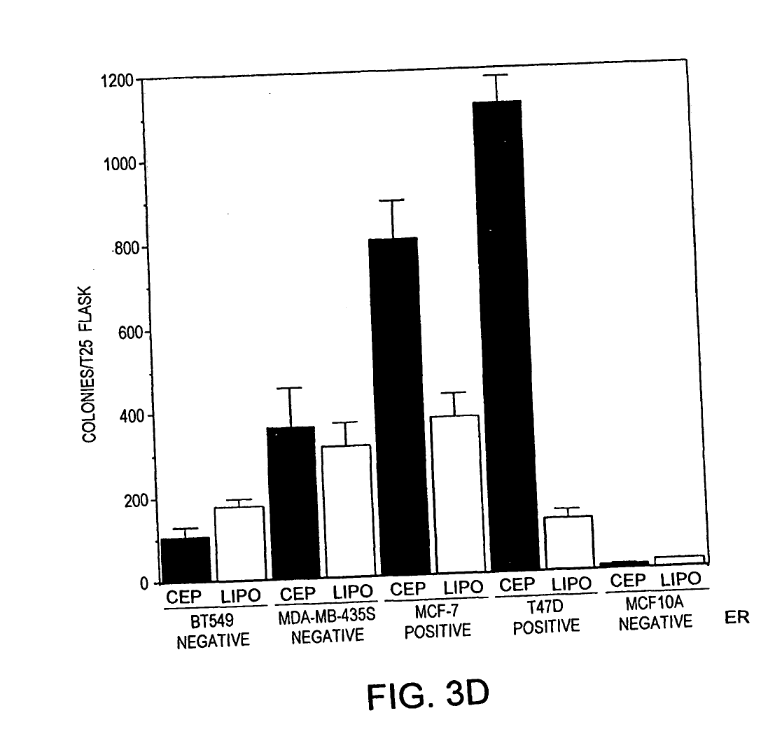

Although the SAGE analysis indicated that estrogen has a different effect in normal and cancerous breast epithelium, it is possible that some of the differences seen were due to the procedure used to isolate these cells. Therefore, to further investigate the genes corresponding to tags that were found to be highly abundant in the SAGE analysis of NER+ cells, their expression levels were examined by northern blot analysis using RNA isolated from multiple separate normal breast organoid preparations and from breast cancer cell lines (FIG. 3A). Organoids are breast ducts composed of luminal and myoepithelial cells with a fraction of luminal epithelial cells expressing the ER+. The northern blot experiments showed that many of the genes identified by the SAGE analysis were highly expressed in normal mammary organoids but not in ER+ breast cancer cell lines (FIG. 3A). The expression of genes that SAGE predicted to be (a) expressed in ER+ breast cancers but not in NER+ cells, or (b) expressed in both cell types was also analyzed The former genes included trefoil factors (pS) 2 and 3, intestinal cysteine rich protein 1, and fatty acid synthase and the latter ones included heat-shock proteins 10 and 27 (Hsp 10 and Hsp27), and Mat-8 phospholemma like protein (FIG. 3A). The northern blot results confirmed that ER+ cells from normal and cancerous mammary epithelium each express a unique set of genes that could explain their differing response to estrogen.

To further investigate the link between estrogen signaling and the expression of some of the genes highly abundant in NER+ cells, the expression pattern of their orthologs in mammary glands of virgin, ovariectomized untreated and estrogen treated, and lactating mice was examined (FIG. 3B). The expression of lipocalin 2 (24p3) and, to a lesser degree, S100A2 appeared to be dependent on the presence of estrogen and lipocalin 2 mRNA and was highly induced in lactating mouse mammary gland (FIG. 3B). Thus, at least some of these genes could be in vivo targets of ER in the normal mammary gland.

To definitively prove that at least some of the genes are expressed in ER+ cells in vivo, mRNA in situ hybridization studies were performed with normal human breast tissue and digitonin labeled riboprobes [St. Croix et al. (2000) Science 289:1197-1202]. A strong hybridization signal was obtained in a fraction of luminal epithelial cells using anti-sense lipocalin 2 and S100A2 probes, whereas hybridization with sense probes gave no or a much fainter background signal (FIG. 3C). Immunohistochemical analysis of adjacent sections using an ERα-specific antibody confirmed that both lipocalin 2 and S100A2 are expressed in ER+ cells (FIG. 3C). Thus, both genes and ERα are indeed co-expressed in a subset of luminal mammary epithelial cells.

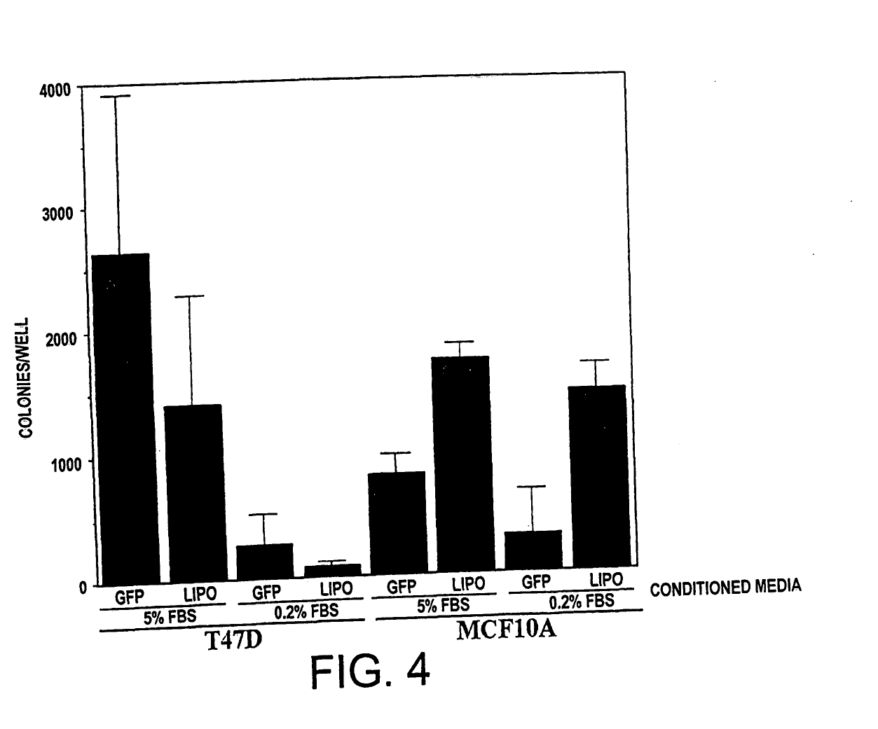

Lipocalin 2 is a secreted protein and could thus be a paracrine factor expressed by NER+ cells that affects the surrounding mammary epithelial cells. In order to determine the effect of lipocalin 2 expression on mammary cell growth, colony growth assays were performed in various ER− and ER+ breast cancer cell lines and in MCF10A cells (FIG. 3D). Lipocalin 2 expression dramatically suppressed colony numbers in the ER+ and breast cancer cell lines tested. In the ER− breast cancer cell line BT549, lipocalin 2 expression caused a small but significant increase in colony numbers. In MCF10A immortalized ER− normal mammary epithelial cells a small increase in the number of colonies (18 vs. 7 colonies) was also observed following lipocalin 2 transfection (FIG. 3D). Since lipocalin 2 is a secreted protein it could act in an autocrine and/or paracrine manner. To determine if exogenous treatment with lipocalin 2 would have similar effects to that of lipocalin 2 overexpression, T47D and MCF10A cells were cultured with conditioned medium obtained from recombinant CHO cells expressing either GFP or lipocalin 2. Similar to lipocalin 2 overexpression, exogenous addition of lipocalin 2 to T47D and MCF10A cells inhibited and enhanced their growth, respectively (FIG. 4). These findings are consistent with a paracrine mechanism of lipocalin 2 action. Thus, in summary, it appears that lipocalin 2 caused an increase in colony numbers in ER− cells and a decrease in colony numbers in ER+ cells, at least ER+ breast cancer cells. These regulatory activities of lipocalin 2 on cell colony formation could be due to its effect on cell proliferation and/or cell survival.

A number of embodiments of the invention have been described. Nevertheless, it will be understood that various modifications may be made without departing from the spirit and scope of the invention. Accordingly, other embodiments are within the scope of the following claims.

1. A reporter construct comprising:
(a) an estrogen response segment comprising five or more estrogen response elements (ERE);
(b) a promoter segment comprising at least one promoter nucleic acid sequence; and
(c) a nucleotide sequence that encodes a reporter polypeptide,
wherein the nucleotide sequence is operably linked to the promoter segment and the estrogen response segment.

2. The reporter construct of claim 1,wherein one or more of the ERE is an ERE from the rat progesterone receptor promoter.

3. The reporter construct of claim 1, wherein the promoter nucleic acid sequence is the distal promoter of the rat progesterone receptor gene.

4. The reporter construct of claim 1, wherein the reporter polypeptide is green fluorescent protein (GFP) or a functional fragment of GFP.

5. The reporter construct of claim 1, wherein the reporter polypeptide is luciferase or a functional fragment of luciferase.

6. A vector comprising the reporter construct of claim 1.

7. The vector of claim 6, wherein the vector is a plasmid.

8. The vector of claim 6, wherein the vector is a viral vector.

9. The vector of claim 8, wherein the viral vector is an adenoviral vector.

10. A cell comprising the vector of claim 6.

11. A method of identifying an estrogen-responsive cell, the method comprising:
(a) introducing the vector of claim 6 into a test cell;
(b) contacting the test cell with estrogen; and
(c) determining whether the test cell expresses the reporter polypeptide.

12. A method of isolating an estrogen-responsive cell, the method comprising:
(a) introducing the vector of claim 6 into a plurality of cells;
(b) contacting the test cell with estrogen; and
(c) isolating a cell that expresses the reporter polypeptide.

13. A method of inhibiting the proliferation or survival of an estrogen-responsive cancer cell in a mammalian subject, the method comprising (a) identifying a mammalian subject as having an estrogen-responsive cancer cell and (b) administering to the subject a lipocalin 2 polypeptide or a DNA that encodes a lipocalin 2 polypeptide.

14. The method of claim 13, wherein the estrogen-responsive cancer cell is a breast cancer cell.

15. The method of claim 13, wherein the mammalian subject is a human patient.

16. The method of claim 13, wherein the administering comprises:
(a) providing a recombinant cell that is a progeny of a cell obtained from the mammal and has been transfected or transformed ex vivo with a DNA encoding a lipocalin 2 polypeptide; and
(b) admnistering the cell to the mammal.

17. An in vitro method of inhibiting the proliferation or survival of an estrogen-responsive cancer cell the method comprising incubating the estrogen-responsive cancer cell in a culture medium comprising an isolated lipocalin 2 polypeptide.

18. The method of claim 17, wherein the estrogen-responsive cancer cell is a breast cancer cell.

19. The reporter construct of claim 1, wherein the estrogen response segment comprises ten or more ERE.

20. The reporter construct of claim 19, wherein the estrogen response segment comprises 20 or more ERE.

21. The reporter construct of claim 20, wherein the estrogen response segment comprises 25 or more ERE.

22. The reporter construct of claim 21, wherein the estrogen response segment comprises 25 ERE.

23. A method of enhancing the proliferation or survival of an estrogen-responsive cancer cell in a mammalian subject, the method comprising (a) identifying a mammalian subject as having a deficit of estrogen-non-responsive normal cells and (b) administering to the subject a lipocalin 2 polypeptide or a DNA that encodes a lipocalin 2 polypeptide.

24. The method of claim 23, wherein the estrogen-non-responsive normal cells are normal mammary cells.

25. The method of claim 23, wherein the mammalian subject is a human subject.

26. The method of claim 23, wherein the administering comprises:
(a) providing a recombinant cell that is a progeny of a cell obtained from the mammal subject and has been transfected or transformed ex vivo with a DNA encoding a lipocalin 2 polypeptide, and
(b) administering the cell to the mammalian subject.

27. An in vitro method of enhancing the proliferation or survival of an estrogen-non-responsive cell, the method comprising incubating the estrogen-non-responsive cell in a culture medium comprising an isolated lipocalin 2 polypeptide.

28. The method of claim 27, wherein the estrogen-non-responsive cell is a normal mammary cell.
